(19) United States
(12) Patent Application Publication
(10) Pub. No.: US 2015/0252108 A1
(43) Pub. Date: Sep. 10, 2015

(54) GLYCOPROTEIN PREPARATIONS

(71) Applicant: MOMENTA PHARMACEUTICALS, INC., Cambridge, MA (US)

(72) Inventors: Nathaniel J. Washburn, Littleton, MA (US); Jonathan C. Lansing, Reading, MA (US); Carlos J. Bosques, Arlington, MA (US); Naveen Bhatnagar, Framingham, MA (US); Dorota A. Bulik, Malden, MA (US); Lynn Markowitz, Waltham, MA (US); Sandra Sipsey, Lawrence, MA (US)

(73) Assignee: Momenta Pharmaceuticals, Inc., Cambridge, MA (US)

(21) Appl. No.: 14/431,217

(22) PCT Filed: Sep. 25, 2013
(86) PCT No.: PCT/US2013/061541

§ 371 Date: Mar. 25, 2015

Related U.S. Application Data
(60) Provisional application No. 61/706,072, filed on Sep. 26, 2012.

(51) Int. Cl. C07K 16/28; G01N 33/566

(52) U.S. Cl. CPC C07K 16/283; G01N 33/566; C07K 2317/41; G01N 2500/04

(57) ABSTRACT

Preparations of glycoproteins, e.g., therapeutic preparations of glycoproteins, having altered levels of affinity for Fcγ receptors relative to reference glycoprotein preparations, and methods of making and methods of using such preparations, are described.

### CROSS REFERENCE TO RELATED APPLICATIONS

This application claims the benefit of U.S. Provisional Application No. 61/706,072, filed on Sep. 26, 2012, the contents of which are herein incorporated by reference in their entirety.

### FIELD OF THE INVENTION

The invention relates generally to glycobiology and glycoproteins.

### BACKGROUND

Therapeutic glycoproteins are an important class of therapeutic biotechnology products, and therapeutic antibodies (including murine, chimeric, humanized and human antibodies and fragments thereof) account for the majority of therapeutic biologic products.

### SUMMARY

The invention encompasses the discovery that particular glycoforms of Fc-containing glycoproteins (e.g., IVIg, therapeutic antibodies of class IgG1, IgG2, IgG3 or IgG4, or Fc-receptor fusion proteins) have specific binding affinities for particular glycoforms of FcγRs (e.g., FcγRIIIa). Accordingly, in some aspects, the invention features glycoprotein compositions comprising a glycoform comprising an Fc region comprising a predetermined or target glycan profile, e.g., a predetermined or target level of glycans selected from the group consisting of a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, a fucosylated glycan, a sulfated glycan, and combinations thereof; which composition is characterized in that, when it is contacted with an FcR, e.g., an FcR described herein, e.g., a FcγIIIa receptor, having a terminal glycan selected from the group consisting of mannose, N-acetylglucosamine, beta-1,4 galactose, and sialic acid, the composition shows an altered affinity (e.g., a higher or a lower affinity) for the FcR (e.g., FcγIIIa receptor) as compared with a reference glycoprotein composition lacking the glycoform.

In some embodiments, the reference glycoprotein composition has a level of glycans that is higher than the predetermined or target level. In some embodiments, the reference glycoprotein composition has a level of glycans that is lower than the predetermined or target level.

In some embodiments, the composition comprises a second glycoform comprising a second predetermined or target level of glycans selected from the group consisting of a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, a fucosylated glycan, a sulfated glycan, and combinations thereof. In some embodiments, the composition is characterized in that, when it is contacted with an FcR, e.g., a FcγIIIa receptor, having a terminal glycan selected from the group consisting of mannose, N-acetylglucosamine, beta-1,4 galactose, and sialic acid, the composition shows an altered affinity for the FcγIIIa receptor as compared with a reference glycoprotein composition lacking one or both of the first and second glycoforms.

In another aspect, the invention features a glycoprotein composition comprising a glycoform comprising an immunoglobulin Fc region comprising a predetermined or target level of fucosylated glycan, which composition is characterized in that, when it is contacted with an FcγIIIa receptor, the composition shows a higher affinity for the FcγIIIa receptor as compared with a reference glycoprotein composition having (a) a lower level of the glycoform or (b) a lower level of fucosylated glycan on the glycoform.

In some embodiments, the FcγIIIa receptor comprises a terminal glycan selected from the group consisting of mannose, N-acetylglucosamine, beta-1,4 galactose, and sialic acid.

In another aspect, the invention features a method of producing a therapeutic preparation, comprising: providing or obtaining an analysis of one or more glycans of an FcR (e.g., an FcγIIIa receptor); and if the one or more glycans comprise a terminal mannose, a terminal N-acetylglucosamine, a terminal beta-1,4 galactose, or a terminal sialic acid, producing a therapeutic preparation comprising a glycoform comprising an immunoglobulin Fc region comprising a predetermined or target level of glycans selected from the group consisting of a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, a sulfated glycan, a fucosylated glycan, and combinations thereof, thereby producing a therapeutic preparation.

In another aspect, the invention features a method of producing a therapeutic preparation, comprising: analyzing one or more glycans of an FcR (e.g., an FcγIIIa receptor); providing a composition of glycoproteins comprising an immunoglobulin Fc region comprising glycans; and if the one or more glycans of the receptor comprise a terminal mannose, a terminal N-acetylglucosamine, a terminal beta-1,4 galactose, or a terminal sialic acid, enriching the composition for a glycoform comprising a predetermined or target level of glycans selected from the group consisting of a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, a sulfated glycan, a fucosylated glycan, and combinations thereof, thereby producing a therapeutic preparation.

In another aspect, the invention features a method of selecting a subject for treatment with a therapeutic preparation, comprising: isolating a cell comprising an FcR (e.g., an FcγIIIa receptor) from a biological sample of the subject (e.g., a blood sample, e.g., a blood cell); analyzing one or more glycans of the FcR (e.g., FcγIIIa receptor); and selecting the subject for treatment with a therapeutic preparation if the one or more glycans comprise a terminal mannose, a terminal N-acetylglucosamine, a terminal beta-1,4 galactose, or a terminal sialic acid; wherein the therapeutic preparation comprises a glycoform comprising an immunoglobulin Fc region comprising a predetermined or target level of glycans selected from the group consisting of a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, a sulfated glycan, a fucosylated glycan, and combinations thereof.

In another aspect, the invention features a method of treating a subject, comprising: isolating a cell comprising an FcR (e.g., an FcγIIIa receptor) from a biological sample of the subject; analyzing one or more glycans of the FcR (e.g., FcγIIIa receptor); and treating the subject with a therapeutic preparation if the one or more glycans comprise a terminal mannose, a terminal N-acetylglucosamine, a terminal beta-1,4 galactose, or a terminal sialic acid, wherein the therapeutic preparation comprises a glycoform comprising an immunoglobulin Fc region comprising a predetermined or target level of glycans selected from the group consisting of a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, a sulfated glycan, a fucosylated glycan, and combinations thereof.

In another aspect, the invention features a glycoprotein composition comprising a glycoform comprising an Fc region comprising a predetermined or target level of sialylated glycan (e.g., a branched glycan having a sialic acid on one or both of an α1,3 arm and an α1,6 arm (e.g., with a NeuAc-α2,6-Gal terminal linkage)), which composition is characterized in that, when it is contacted with an FcγIIIa receptor having a terminal glycan selected from the group consisting of mannose, N-acetylglucosamine, beta-1,4 galactose, or sialic acid, the composition shows a higher affinity for the FcγIIIa receptor as compared with a reference glycoprotein composition having (a) a lower level of the glycoform or (b) a lower level of sialylated glycan (e.g., a branched glycan having a sialic acid on one or both of an α1,3 arm and an α1,6 arm (e.g., with a NeuAc-α2,6-Gal terminal linkage)) on the glycoform.

In another aspect, the invention features a glycoprotein composition comprising a glycoform comprising an Fc region comprising a predetermined or target level of sialylated glycan (e.g., a branched glycan having a sialic acid on one or both of an α1,3 arm and an α1,6 arm (e.g., with a NeuAc-α2,6-Gal terminal linkage)), which composition is characterized in that, when it is contacted with a population of FcγIIIa receptors comprising glycans, the composition shows an altered, e.g., a higher, affinity for an FcγIIIa receptor comprising a terminal N-acetylglucosamine as compared with an Fc receptor comprising a terminal mannose or a terminal beta-1,4 galactose.

In some embodiments, the predetermined or target level of sialylated glycan (e.g., a branched glycan having a sialic acid on one or both of an α1,3 arm and an α1,6 arm (e.g., with a NeuAc-α2,6-Gal terminal linkage)) is about 30%, 40%, 50%, 60%, 70%, 80%, 90%, or 100%.

In another aspect, the invention features a glycoprotein composition comprising a glycoform comprising an Fc region comprising a predetermined or target level of sialylated glycan (e.g., a branched glycan having a sialic acid on one or both of an α1,3 arm and an α1,6 arm (e.g., with a NeuAc-α2,6-Gal terminal linkage)), which composition is characterized in that, when it is contacted with a population of FcγIIIa receptors comprising glycans, the composition shows an altered, e.g., a lower, affinity for an FcγIIIa receptor comprising a terminal mannose as compared with an Fc receptor comprising a terminal beta-1,4 galactose or a terminal N-acetylglucosamine.

In some embodiments, the predetermined or target level of sialylated glycan (e.g., a branched glycan having a sialic acid on one or both of an α1,3 arm and an α1,6 arm (e.g., with a NeuAc-α2,6-Gal terminal linkage)) is about 0%, 5%, 10%, 15%, 20%, 25%, or about 30%.

In another aspect, the invention features a glycoprotein composition comprising a glycoform comprising an Fc region comprising a predetermined or target level of sialylated glycan (e.g., a branched glycan having a sialic acid on one or both of an α1,3 arm and an α1,6 arm (e.g., with a NeuAc-α2,6-Gal terminal linkage)), which composition is characterized in that, when it is contacted with a population of FcγIIIa receptors comprising glycans, the composition shows an altered, e.g., a lower, affinity for an FcγIIIa receptor comprising a terminal beta-1,4 galactose as compared with an Fc receptor comprising a terminal N-acetylglucosamine.

In another aspect, the invention features a method of producing a preparation of glycoproteins, comprising: providing a plurality of Fcγ receptors; determining binding of a reference glycoprotein preparation to the plurality of receptors to obtain a reference binding profile; producing a glycoprotein preparation comprising a plurality of glycoproteins; determining binding of the glycoprotein preparation to the plurality of Fcγ receptors to obtain a preparation binding profile; and formulating the preparation into a drug product if the preparation binding profile is at least about 50%, 60%, 70%, 80%, 85%, 90%, 95%, or is 100% identical to the reference binding profile.

In some embodiments, the plurality of Fcγ receptors are provided on an array. In some embodiments, the array comprises one or more of FcγRI, FcγRIIA, FcγRIIB, FcγRIIIA, FcγRIIIB, FcγRIV, and FcRn receptors. In some embodiments, the array comprises FcγRIIIA receptors comprising terminal glycans selected from the group consisting of a mannose, a N-acetylglucosamine, a beta-1,4 galactose and a sialic acid.

In some embodiments, the reference binding profile comprises binding affinities of the reference glycoprotein preparation to one or more FcγRIIIA receptors comprising terminal glycans selected from the group consisting of a mannose, a N-acetylglucosamine, a beta-1,4 galactose, and a sialic acid, and the preparation binding profile comprises binding affinities of the glycoprotein preparation to the one or more FcγRIIIA receptors.

In another aspect, the invention features a method of manufacturing a glycoprotein drug product, comprising: providing or obtaining a test glycoprotein preparation; acquiring a binding profile of the test glycoprotein preparation for a plurality of FcRs (e.g., a plurality of FcR glycoforms, e.g., a plurality of FcγRIII glycoforms); and processing at least a portion of the test glycoprotein preparation into a drug product if the binding profile of the test glycoprotein preparation meets a reference binding profile (e.g., binding profile of a reference glycoprotein preparation described herein), thereby manufacturing a drug product.

In some embodiments, the reference binding profile is a binding profile of an FDA approved Fc-containing therapeutic glycoprotein preparation (e.g., an FDA approved Fc-containing therapeutic glycoprotein preparation described herein).

In another aspect, the invention features a method of manufacturing a glycoprotein drug product, comprising: providing a host cell that is genetically engineered to express a recombinant Fc region-containing glycoprotein; culturing the host cell under conditions whereby the cell expresses the recombinant Fc region-containing glycoprotein; harvesting the recombinant Fc region-containing glycoprotein from the host cell culture to produce a test glycoprotein preparation; acquiring a binding profile of the test glycoprotein preparation for a plurality of FcRs (e.g., a plurality of FcR glycoforms, e.g., a plurality of FcγRIII glycoforms); and processing or directing the processing of at least a portion of the test glycoprotein preparation as a drug product if the preparation meets a reference binding profile (e.g., a binding profile of a reference glycoprotein preparation), thereby manufacturing a drug product.

In another aspect, the invention features a method of identifying a patient who has been diagnosed with a disease, for treatment with a therapeutic, Fc region-containing glycoprotein preparation that is approved for treatment of the disease, wherein the improvement comprises: analyzing one or more FcγR glycans from a biological sample of the patient, and identifying the patient for treatment with the preparation if the FcγR glycans match a reference glycan profile.

In any of the aspects described, a glycoprotein preparation (e.g., a therapeutic glycoprotein preparation) can include at least 5%, 10%, 15%, 20%, 25%, 30%, 35%, 40%, 45%, 50%, 55%, 60%, 65%, 70%, 75%, 80%, 85%, 90%, 95%, or can include 100% of a particular glycoform (e.g., a glycoform with a predetermined glycan profile as described herein).

In any of the aspects described herein, a predetermined or target level can be a level of glycans on an isolated glycoform or a level of glycans on a plurality of a particular glycoform.

In any of the aspects described herein, a predetermined or target level of glycans can be one or more of the following:

(a) about 10%, 20%, 30%, 40%, 50%, 60%, 70%, 80%, 90%, or 100% high mannose glycans (e.g., Man-3, Man-4, Man-5, Man-6, Man-7, and/or Man-8);

(b) about 10%, 20%, 30%, 40%, 50%, 60%, 70%, 80%, 90%, or 100% sialylated glycans (e.g., monosialylated on an α1-3 arm of a branched glycan (e.g., with a NeuAc-α2,6-Gal terminal linkage), monosialylated on an α1-6 arm of a branched glycan (e.g., with a NeuAc-α2,6-Gal terminal linkage), and/or disialylated on an α1-3 arm and an α1-6 arm (e.g., with a NeuAc-α2,6-Gal terminal linkage));

(c) about 10%, 20%, 30%, 40%, 50%, 60%, 70%, 80%, 90%, or 100% galactosylated glycans (e.g., monogalactosylated on an α1-3 arm of a branched glycan, monogalactosylated on an α1-6 arm of a branched glycan, digalactosylated on an α1-3 arm and an α1-6 arm, and/or having a Gal-α1,3-Gal terminal linkage);

(d) about 10%, 20%, 30%, 40%, 50%, 60%, 70%, 80%, 90%, or 100% glycans comprising a terminal N-acetylglucosamine (e.g., on one or both arms of a branched glycan) and/or a bisecting N-acetylglucosamine;

(e) about 10%, 20%, 30%, 40%, 50%, 60%, 70%, 80%, 90%, or 100% fucosylated glycans;

(f) about 10%, 20%, 30%, 40%, 50%, 60%, 70%, 80%, 90%, or 100% sulfated glycans; and

(g) about 10%, 20%, 30%, 40%, 50%, 60%, 70%, 80%, 90%, or 100% hybrid glycans (e.g., fucosylated hybrid glycans and/or afucosylated hybrid glycans).

In any of the aspects described herein, a predetermined or target level can lack one or more of these levels of glycans. For example, a predetermined or target level can include a certain level of one or more glycans and lack other glycan(s).

In some embodiments, the predetermined or target level is a substantial amount of a combination of galactosylated glycans and high mannose glycans (e.g., about 50% galactosylated glycans and about 50% high mannose glycans). In some embodiments, the predetermined or target level is about 10%, 20%, 30%, 40%, 50%, 60%, 70%, 80%, or 90% galactosylated glycans (e.g., monogalactosylated on an α1-3 arm of a branched glycan, monogalactosylated on an α1-6 arm of a branched glycan, digalactosylated on an α1-3 arm and an α1-6 arm, and/or having a Gal-α1,3-Gal terminal linkage); and about 90%, 80%, 70%, 60%, 50%, 40%, 30%, 20%, or 10% high mannose glycans (e.g., Man-3, Man-4, Man-5, Man-6, Man-7, or Man-8).

In some embodiments, the predetermined or target level is a substantial amount of a combination of afucosylated glycans and galactosylated glycans (e.g., about 50% afucosylated glycans and about 50% galactosylated glycans). In some embodiments, the predetermined or target level is about 10%, 20%, 30%, 40%, 50%, 60%, 70%, 80%, or 90% afucosylated glycans; and about 90%, 80%, 70%, 60%, 50%, 40%, 30%, 20%, or 10% galactosylated glycans (e.g., monogalactosylated on an α1-3 arm of a branched glycan, monogalactosylated on an α1-6 arm of a branched glycan, digalactosylated on an α1-3 arm and an α1-6 arm, and/or having a Gal-α1,3-Gal terminal linkage).

In some embodiments, the predetermined or target level is a substantial amount of a combination of afucosylated glycans, galactosylated glycans, and high mannose glycans (e.g., about 33% of each of afucosylated glycans, galactosylated glycans, and high mannose glycans). In some embodiments, the predetermined or target level is about 10%, 20%, 30%, 40%, 50%, 60%, 70%, 80%, or 90% afucosylated glycans; about 10%, 20%, 30%, 40%, 50%, 60%, 70%, 80%, or 90% galactosylated glycans (e.g., monogalactosylated on an α1-3 arm of a branched glycan, monogalactosylated on an α1-6 arm of a branched glycan, digalactosylated on an α1-3 arm and an α1-6 arm, and/or having a Gal-α1,3-Gal terminal linkage); and about 10%, 20%, 30%, 40%, 50%, 60%, 70%, 80%, or 90% high mannose glycans (e.g., Man-3, Man-4, Man-5, Man-6, Man-7, or Man-8).

In some embodiments, the predetermined or target level is a substantial amount of a combination of sialylated glycans and sulfated glycans (e.g., about 50% sialylated glycans and 50% sulfated glycans). In some embodiments, the predetermined or target level is about 10%, 20%, 30%, 40%, 50%, 60%, 70%, 80%, or 90% sialylated glycans (e.g., monosialylated on an α1-3 arm of a branched glycan (e.g., with a NeuAc-α2,6-Gal terminal linkage), monosialylated on an α1-6 arm of a branched glycan (e.g., with a NeuAc-α2,6-Gal terminal linkage), and/or disialylated on an α1-3 arm and an α1-6 arm (e.g., with a NeuAc-α2,6-Gal terminal linkage)); and about 90%, 80%, 70%, 60%, 50%, 40%, 30%, 20%, or 10% sulfated glycans.

In some embodiments, the predetermined or target level is a substantial amount of a combination of N-acetylglucosamine and sulfated glycans (e.g., about 50% N-acetylglucosamine and 50% sulfated glycans). In some embodiments, the predetermined or target level is about 10%, 20%, 30%, 40%, 50%, 60%, 70%, 80%, or 90% terminal N-acetylglucosamine (e.g., on one or both arms of a branched glycan) and/or a bisecting N-acetylglucosamine; and about 90%, 80%, 70%, 60%, 50%, 40%, 30%, 20%, or 10% sulfated glycans.

In some embodiments, the predetermined or target level is a substantial amount of a combination of N-acetylglucosamine, sulfated glycans, and sialylated glycans (e.g., about 33% of each of N-acetylglucosamine, sulfated glycans, and sialylated glycans). In some embodiments, the predetermined or target level is about 10%, 20%, 30%, 40%, 50%, 60%, 70%, 80%, or 90% terminal N-acetylglucosamine (e.g., on one or both arms of a branched glycan) and/or a bisecting N-acetylglucosamine; about 90%, 80%, 70%, 60%, 50%, 40%, 30%, 20%, or 10% sulfated glycans; and about 10%, 20%, 30%, 40%, 50%, 60%, 70%, 80%, or 90% sialylated glycans (e.g., monosialylated on an α1-3 arm of a branched glycan (e.g., with a NeuAc-α2,6-Gal terminal linkage), monosialylated on an α1-6 arm of a branched glycan (e.g., with a NeuAc-α2,6-Gal terminal linkage), and/or disialylated on an α1-3 arm and an α1-6 arm (e.g., with a NeuAc-α2,6-Gal terminal linkage)).

In any of the aspects described herein, a composition or preparation comprising a glycoform comprising a predetermined or target level of one or more glycans, when contacted with one or more of FcγRI, FcγRIIA, FcγRIIB, FcγRIIIA, FcγRIIIB, FcγRIV, and FcRn, having a terminal glycan selected from the group consisting of mannose, N-acetylglucosamine, beta-1,4 galactose, and sialic acid, the composition or preparation shows an altered affinity (e.g., a higher or a lower affinity) for the one or more of FcγRI, FcγRIIA, FcγRIIB, FcγRIIIA, FcγRIIIB, FcγRIV, and FcRn, as compared with a reference glycoprotein composition or preparation lacking the glycoform. In some embodiments, the composition or preparation has a different, e.g., a detectably higher or lower, activity, such as an immune cell activating activity, e.g., Fc receptor affinity, Fc receptor specificity, complement activation activity, signaling activity, targeting activity, effector function, half-life, clearance, pro-inflammatory, anti-inflammatory, or transcytosis activity than a reference glycoprotein composition or preparation lacking the glycoform. In some embodiments, the effector function is antibody dependent cellular cytotoxicity, complement dependent cytotoxicity, programmed cell death, or cellular phagocytosis.

In some aspects, a therapeutic composition described herein comprises a glycoform having a predetermined or target level described herein. In some embodiments, a therapeutic composition comprises about 10%, 20%, 30%, 40%, 50%, 60%, 70%, 80%, 90%, or 100% of a glycoform described herein, e.g., a glycoform having a predetermined or target level described herein.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is a schematic illustration of an IgG antibody molecule.

FIG. 2 are diagrammatic representations of relative abundance of glycans at Asn162 of control srhFcγRIIIa receptor and receptor glycovariants R3-R5.

FIG. 3 is a diagrammatic representation of relative abundances of glycans of IgG1 Fc and IgG1 sialylated Fc, depicting structures for major species.

FIG. 4A is a diagrammatic representation of KD values for Fc (non-sialylated) binding to control srhFcγRIIIa and to receptor glycovariants R3-R5. FIG. 4B is a diagrammatic representation of KD values for Fc (sialylated) binding to control srhFcγRIIIa and to receptor glycovariants R3-R5.

FIG. 5 is a diagrammatic representation of relative abundances of glycans from rituximab glycoform preparations or control rituximab.

FIG. 6 is a diagrammatic representation of relative ADCC activity of NK cells from various donors induced by rituximab glycoform preparations or control rituximab.

FIG. 7 are diagrammatic representations showing ADCC activity of NK cells from donor 1 (FIG. 7A) or donor 2 (FIG. 7B) induced by afucosylated rituximab preparation or control rituximab.

FIG. 8 is a diagrammatic representation of relative abundance of Asn297 glycans from rFc, s1-rFc, and s2-rFc.

FIG. 9A is a diagrammatic representation of relative abundance of Asn297 glycans from FcγRIIIa receptors expressed in HEK and CHO cells; and FIG. 9B is a diagrammatic representation of relative abundance of Asn297 glycans from FcγRIIIa receptors expressed in NS0 cells. Most likely structures shown based on mass and MS/MS fragmentation. Composition shown in axis title is expressed as (HexNAc,Hex,Fuc,NeuAc,NeuGc).

FIG. 10 is a diagrammatic representation of affinities of recombinant Fc (rFc) for FcγRIIIa (Val176 isoform) expressed in HEK, CHO, or NS0 cells.

FIG. 11 is a diagrammatic representation of affinities of recombinant Fc (rFc) for FcγRIIIa (Val176 isoform) expressed in CHO cells before and after desialylation.

FIG. 12 are diagrammatic representations of affinities of rFc, s1rFc and s2rFc with FcγRIIIa (Val176 isoform) expressed in HEK, CHO, or NS0 cells.

FIG. 13 are diagrammatic representations of affinity of soluble rhFcγRIIIa expressed in HEK cells and of relative affinity (EC50) of FcγRIIIa expressed on the surface of HEK cells.

FIG. 14 are diagrammatic representations of affinities of rFc, s1rFc and s2rFc with FcγRIIIa polymorphic variants (Val176 or Phe176) expressed in HEK cells.

FIG. 15 are diagrammatic representations of affinities of different Fc glycoforms for FcγRIIIa Val176 isoform expressed in CHO cells before and after desialylation.

### DETAILED DESCRIPTION

The invention encompasses the discovery that particular glycoforms of Fc-containing glycoproteins (e.g., IVIg, therapeutic antibodies of class IgG1, IgG2, IgG3 or IgG4, or Fc-receptor fusion proteins) have specific binding affinities for particular glycoforms of FcγRs (e.g., FcγRIIIa). Such glycoproteins are a growing class of biotherapeutic drugs in the pharmaceutical market. These drugs comprise a mixture of isoforms comprising many different molecules that may have a range of pharmacokinetic, pharmacodynamic, efficacy, and/or safety profiles. The discovery of specific FcγR-IgG glycoform interactions is unexpected and useful, e.g., in designing glycovariants of therapeutic biologic products that are more suited for specific patient populations, allowing specific glycovariants to be rationally designed for a particular population of patients (e.g., expressing particular FcγR glycoforms). In addition, the invention is useful in diagnostic tools for selecting specific patient populations with specific Fc receptor glycoforms for more suitable treatments with glycoengineered Fc-containing glycoproteins. Further, a disease or condition that is associated with a particular change in glycosylation (e.g., FcγR glycosylation) can be treated with an appropriately glycoengineered Fc-containing glycoprotein.

The constant regions (Fc regions) of antibodies interact with cellular binding partners to mediate antibody function and activity, such as antibody-dependent effector functions and complement activation. For IgG type antibodies, binding sites for complement C1q and Fc receptors (FcγRs) are located in the CH2 domain of the Fc region.

Coexpression of activating and inhibitory FcRs on different target cells modulates antibody-mediated immune responses. In addition to their involvement in the efferent phase of an immune response, FcRs are also important for regulating B cell and dendritic cell (DC) activation. For example, in the case of IgG type antibodies, different classes of FcγR mediate various cellular responses, such as phagocytosis by macrophages, antibody-dependent cell-mediated cytotoxicity by NK cells, and degranulation of mast cells. Each FcγR displays different binding affinities and IgG subclass specificities. Lectin receptors also play a role. For example, Dc-SIGN has been shown to play a role in the anti-inflammatory activity of Fc, e.g., in IVIG (see, e.g., WO2008057634; WO2009132130).

Antibodies are glycosylated at conserved positions in the constant regions of their heavy chain. For example, IgG antibodies have a single N-linked glycosylation site at Asn297 of the CH2 domain. Each antibody isotype has a distinct variety of N-linked carbohydrate structures in the constant regions. For human IgG, core oligosaccharides normally consists of GlcNAc₂Man₃GlcNAc, with differing numbers of outer residues. Variation among individual IgG's can occur via attachment of galactose and/or galactose-sialic acid at two terminal GlcNAc or via attachment of a third GlcNAc arm (bisecting GlcNAc).

The Fc domain of IgGs interacts with the family of highly glycosylated Fcγ receptors including FcγRIa, FcγRIIa, FcγRIIb, FcγRIIIa and FcγRIIIb. The Fc N-glycans from IgG1 can interact directly with the glycans found at asparagine 162 of the FcγRIIIa receptor (Ferrara et al., Proc. Natl. Acad. Sci. U.S.A. 108:12669-12674 (2011)). The absence of glycosylation at this site abrogates the increased affinity of afucosyl IgG1 for the FcγRIIIa receptor (Ferrara et al., J. Biol. Chem. 281:5032-5036 (2006)). Glycosylation at asparagine 45 of the FcγRIIIa receptor can also influence receptor binding (Shibata-Koyama et al., Glycobiology 19:126-134 (2009)). Further, the FcγRIIIa receptors found on NK cells have a different glycosylation pattern than those found on other cell types, and FcγRIIIa from NK cells have a higher affinity for IgG1 than those found on monocytes (Kimberly et al., J. Immunol. 159:3849-57 (1997)). Given the critical role of the interactions between therapeutic IgGs and FcγRs for mediating therapeutic efficacy, it is desirable to have a better understanding of the mechanism driving the interactions between these two families of molecules.

The inventors have discovered that interactions of Fc-containing glycoproteins and Fcγreceptors are mediated, at least in part, by the glycans on both the glycoproteins and the Fcγreceptors, and that glycoproteins comprising particular glycans interact with Fcγ receptors having particular glycans to mediate specific activities. Described herein are glycoprotein compositions (e.g., therapeutic glycoprotein preparations (e.g., IVIG, antibodies or fusion proteins, such as Fc fusion proteins)) comprising glycoforms having particular glycans that demonstrate particular binding affinities for Fcγ receptors comprising particular glycans, e.g., as compared with reference glycoprotein compositions (e.g., lacking the particular glycans). Methods of making and using such compositions (e.g., therapeutic preparations) are also described. In certain instances, a preparation described herein includes a mixture of two or more particular glycoforms (e.g., each having a predetermined or target level of glycans), specifically engineered to have altered affinity to one or more FcγR, relative to a reference glycoprotein preparation.

### DEFINITIONS

As used herein, “glycan” is a sugar polymer (moiety) component of a glycoprotein. The term “glycan” encompasses free glycans, including glycans that have been cleaved or otherwise released from a glycoprotein. In some embodiments, a glycan is N-linked to an Fc region. In some embodiments, a glycan is released from an N-glycosylation site of an Fc region.

As used herein, the term “glycoprotein” refers to a protein that contains a peptide backbone covalently linked to one or more sugar moieties (i.e., glycans). Sugar moiety(ies) may be in the form of disaccharides, oligosaccharides, and/or polysaccharides. Sugar moiety(ies) may comprise a single unbranched chain of sugar residues or may comprise one or more branched chains. Glycoproteins can contain O-linked sugar moieties and/or N-linked sugar moieties.

By “purified” (or “isolated”) refers to a nucleic acid sequence (e.g., a polynucleotide) or an amino acid sequence (e.g., a polypeptide) that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acid sequences. An isolated nucleic acid sequence or amino acid sequence can be at least 60% free, or at least 75% free, or at least 90% free, or at least 95% free from other components present in natural environment of the indicated nucleic acid sequence or amino acid sequence.

As used herein, “polynucleotide” (or “nucleotide sequence” or “nucleic acid molecule”) refers to an oligonucleotide, nucleotide, or polynucleotide, and fragments or portions thereof, and to DNA or RNA of genomic or synthetic origin, which may be single- or double-stranded, and represent the sense or anti-sense strand.

As used herein, “polypeptide” (or “amino acid sequence” or “protein”) refers to an oligopeptide, peptide, polypeptide, or protein sequence, and fragments or portions thereof, and to naturally occurring or synthetic molecules. “Amino acid sequence” and like terms, such as “polypeptide” or “protein”, are not meant to limit the indicated amino acid sequence to the complete, native amino acid sequence associated with the recited protein molecule.

The articles “a” and “an” are used herein to refer to one or to more than one (i.e., to at least one) of the grammatical object of the article. By way of example, “an element” means one element or more than one element.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

The term “treatment” or “treating”, as used herein, refers to administering a therapy in an amount, manner, and/or mode effective to improve a condition, symptom, or parameter associated with a disorder or condition or to prevent or reduce progression of a disorder or condition, to a degree detectable to one skilled in the art. An effective amount, manner, or mode can vary depending on the subject and may be tailored to the subject.

The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). Light chains of the immunoglobulin can be of types kappa or lambda.

As used herein, the term “constant region” refers to a polypeptide that corresponds to, or is derived from, one or more constant region immunoglobulin domains of an antibody. A constant region can include any or all of the following immunoglobulin domains: a CH1 domain, a hinge region, a CH2 domain, a CH3 domain (derived from an IgA, IgD, IgG, IgE, or IgM), and a CH4 domain (derived from an IgE or IgM).

As used herein, the term “Fc region” refers to a dimer of two “Fc polypeptides”, each “Fc polypeptide” comprising the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or all of the flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIG.

As used herein, the term “Fc region variant” refers to an analog of an Fc region that possesses one or more Fc-mediated activities described herein. This term includes Fc regions comprising one or more amino acid modifications relative to a wild type or naturally existing Fc region. For example, variant Fc regions can possess at least about 50% homology, at least about 75% homology, at least about 80% homology, at least about 85%, homology, at least about 90% homology, at least about 95% homology, or more, with a naturally existing Fc region. Fc region variants also include Fc regions comprising one or more amino acid residues added to or deleted from the N- or C-terminus of a wild type Fc region.

As used herein, the term “glycoprotein preparation” refers to a set of individual glycoprotein molecules, each of which comprises a polypeptide having a particular amino acid sequence (which amino acid sequence includes at least one glycosylation site) and at least one glycan covalently attached to the at least one glycosylation site. Individual molecules of a particular glycoprotein within a glycoprotein preparation typically have identical amino acid sequences but may differ in the occupancy of the at least one glycosylation sites and/or in the identity of the glycans linked to the at least one glycosylation sites. That is, a glycoprotein preparation may contain only a single glycoform of a particular glycoprotein, but more typically contains a plurality of glycoforms. Different preparations of the same glycoprotein may differ in the identity of glycoforms present (e.g., a glycoform that is present in one preparation may be absent from another) and/or in the relative amounts of different glycoforms.

The term “glycoform” is used herein to refer to a particular form of a glycoprotein. That is, when a glycoprotein includes a particular polypeptide that has the potential to be linked to different glycans or sets of glycans, then each different version of the glycoprotein (i.e., where the polypeptide is linked to a particular glycan or set of glycans) is referred to as a “glycoform”.

“Predetermined level” or “target level” as used herein, refers to a pre-specified particular level of one or more particular glycans, e.g., high mannose glycans, sialylated glycans, galactosylated glycans, glycans comprising a terminal N-acetylglucosamine, fucosylated glycans, and/or sulfated glycans. In some embodiments, a predetermined or target level is an absolute value or range. In some embodiments, a predetermined level or target level is a relative value. In some embodiments, a predetermined level or target level is the same as or different (e.g., higher or lower) than a level of one or more particular glycans (e.g., high mannose glycans, sialylated glycans, galactosylated glycans, glycans comprising a terminal N-acetylglucosamine, fucosylated glycans, and/or sulfated glycans) in a reference, e.g., a reference glycoprotein product, or a reference document such as a specification, alert limit, or master batch record for a pharmaceutical product.

In some embodiments, a predetermined level or target level refers to an absolute level of (e.g., number of moles of) one or more glycans (e.g., high mannose glycans, sialylated glycans, galactosylated glycans, glycans comprising a terminal N-acetylglucosamine, fucosylated glycans, and/or sulfated glycans) in a glycoprotein preparation. In some embodiments, a predetermined level or target level refers to a level of one or more glycans (e.g., high mannose glycans, sialylated glycans, galactosylated glycans, glycans comprising a terminal N-acetylglucosamine, fucosylated glycans, and/or sulfated glycans) in a glycoprotein preparation relative to total level of glycans in the glycoprotein preparation. In some embodiments, a predetermined level or target level is expressed as a “percent”.

For any given parameter, “percent” refers to the number of moles of a particular glycan (glycan X) relative to total moles of glycans of a preparation. In some embodiments, “percent” refers to the number of moles of PNGase F-released Fc glycan X relative to total moles of PNGase F-released Fc glycans detected.

“Reference glycoprotein”, as used herein, refers to a glycoprotein having substantially the same amino acid sequence as (e.g., having about 90-100% identical amino acids of) a glycoprotein described herein, e.g., a glycoprotein to which it is compared. In some embodiments, a reference glycoprotein is a therapeutic glycoprotein described herein, e.g., an FDA approved therapeutic glycoprotein.

As used herein, an “N-glycosylation site of an Fc region” refers to an amino acid residue within an Fc region to which a glycan is N-linked.

As used herein, the terms “coupled”, “linked”, “joined”, “fused”, and “fusion” are used interchangeably. These terms refer to the joining together of two more elements or components by whatever means, including chemical conjugation or recombinant means.

The terms “overexpress,” “overexpression” or “overexpressed” interchangeably refer to a protein or nucleic acid that is transcribed or translated at a detectably greater level, such as in a cancer cell, in comparison to a control cell. The term includes expression due to transcription, post transcriptional processing, translation, post-translational processing, cellular localization (e.g., organelle, cytoplasm, nucleus, cell surface), and RNA and protein stability, as compared to a control cell. Overexpression can be detected using conventional techniques, e.g., for detecting mRNA (i.e., RT-PCR, PCR, hybridization) or proteins (i.e., ELISA, immunohistochemical techniques). Overexpression can be expression in an amount greater than about 10%, 20%, 30%, 40%, 50%, 60%, 70%, 80%, 90% or more compared to a control cell. In certain instances, overexpression is 1-fold, 2-fold, 3-fold, 4-fold, or more, higher level of transcription or translation compared to a control cell.

### I. Glycoproteins

The present disclosure encompasses preparations of glycoproteins (e.g., therapeutic preparations of glycoproteins), and methods of making and using such preparations. Glycoproteins include, for example, any of a variety of hematologic agents (including, for instance, erythropoietin, blood-clotting factors, etc.), interferons, colony stimulating factors, antibodies, enzymes, and hormones. The identity of a particular glycoprotein is not intended to limit the present disclosure, and a therapeutic preparation described herein can include any glycoprotein of interest having an Fc region.

A glycoprotein described herein can include a target-binding domain that binds to a target of interest (e.g., binds to an antigen). For example, a glycoprotein, such as an antibody, can bind to a transmembrane polypeptide (e.g., receptor) or ligand (e.g., a growth factor). Exemplary molecular targets (e.g., antigens) for glycoproteins described herein (e.g., antibodies) include CD proteins such as CD2, CD3, CD4, CD8, CD11, CD19, CD20, CD22, CD25, CD33, CD34, CD40, CD52; members of the ErbB receptor family such as the EGF receptor (EGFR, HER1, ErbB1), HER2 (ErbB2), HER3 (ErbB3) or HER4 (ErbB4) receptor; macrophage receptors such as CRIg; tumor necrosis factors such as TNFα or TRAIL/Apo-2; cell adhesion molecules such as LFA-1, Mac1, p150,95, VLA-4, ICAM-1, VCAM and αvβ3 integrin including either α or β subunits thereof (e.g., anti-CD11a, anti-CD18 or anti-CD11b antibodies); growth factors and receptors such as EGF, FGFR (e.g., FGFR3) and VEGF; IgE; cytokines such as IL1; cytokine receptors such as IL2 receptor; blood group antigens; flk2/flt3 receptor; obesity (OB) receptor; mpl receptor; CTLA-4; protein C; neutropilins; ephrins and receptors; netrins and receptors; slit and receptors; chemokines and chemokine receptors such as CCL5, CCR4, CCR5; amyloid beta; complement factors, such as complement factor D; lipoproteins, such as oxidized LDL (oxLDL); lymphotoxins, such as lymphotoxin alpha (LTa). Other molecular targets include Tweak, B7RP-1, proprotein convertase subtilisin/kexin type 9 (PCSK9), sclerostin, c-kit, Tie-2, c-fms, and anti-M1.

Also described herein are glycoprotein preparations (e.g., therapeutic glycoprotein preparations) that have different affinities for Fcγ receptors and/or different levels of effector functions (e.g., an effector function described herein), e.g., as compared with a control or reference glycoprotein preparations. Nonlimiting, exemplary reference glycoprotein preparations can include abatacept (Orencia®, Bristol-Myers Squibb), abciximab (ReoPro®, Roche), adalimumab (Humira®, Bristol-Myers Squibb), alefacept (Amevive®, Astellas Pharma), alemtuzumab (Campath®, Genzyme/Bayer), basiliximab (Simulect®, Novartis), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), and trastuzumab (Herceptin®, Roche).

### A. N-Linked Glycosylation

N-linked oligosaccharide chains are added to a protein in the lumen of the endoplasmic reticulum (see Molecular Biology of the Cell, Garland Publishing, Inc. (Alberts et al., 1994)). Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains 3 glucose, 9 mannose, and 2 N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues.

N-glycans can be subdivided into three distinct groups called “high mannose type”, “hybrid type”, and “complex type”, with a common pentasaccharide core (Man (alpha1,6)-(Man(alpha1,3))-Man(beta1,4)-GlcpNAc(beta 1,4)-GlcpNAc(beta 1,N)-Asn) occurring in all three groups.

After initial processing in the endoplasmic reticulum, the glycoprotein is transported to the Golgi where further processing may take place. If the glycan is transferred to the Golgi before it is completely trimmed to the core pentasaccharide structure, it results in a “high-mannose glycan”.

Additionally or alternatively, one or more monosaccharides units of N-acetylglucosamine may be added to core mannose subunits to form a “complex glycan”. Galactose may be added to N-acetylglucosamine subunits, and sialic acid subunits may be added to galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases, known in the art.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in glycoproteins produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either α2,3 or α2,6.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

N-Linked Glycosylation in Antibodies

Antibodies are glycosylated at conserved, N-linked glycosylation sites in the Fc regions of immunoglobulin heavy chains. For example, each heavy chain of an IgG antibody has a single N-linked glycosylation site at Asn297 of the CH2 domain (see Jefferis, Nature Reviews 8:226-234 (2009)). IgA antibodies have N-linked glycosylation sites within the CH2 and CH3 domains, IgE antibodies have N-linked glycosylation sites within the CH3 domain, and IgM antibodies have N-linked glycosylation sites within the CH1, CH2, CH3, and CH4 domains (see Arnold et al., J. Biol. Chem. 280:29080-29087 (2005); Mattu et al., J. Biol. Chem. 273:2260-2272 (1998); Nettleton et al., Int. Arch. Allergy Immunol. 107:328-329 (1995)).

Each antibody isotype has a distinct variety of N-linked carbohydrate structures in the constant regions. For example, IgG has a single N-linked biantennary carbohydrate at Asn297 of the CH2 domain in each Fc polypeptide of the Fc region, which also contains the binding sites for C1q and FcγR (see Jefferis et al., Immunol. Rev. 163:59-76 (1998); and Wright et al., Trends Biotech 15:26-32 (1997)). For human IgG, the core oligosaccharide normally consists of GlcNAc₂Man₃GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc).

### B. Antibodies

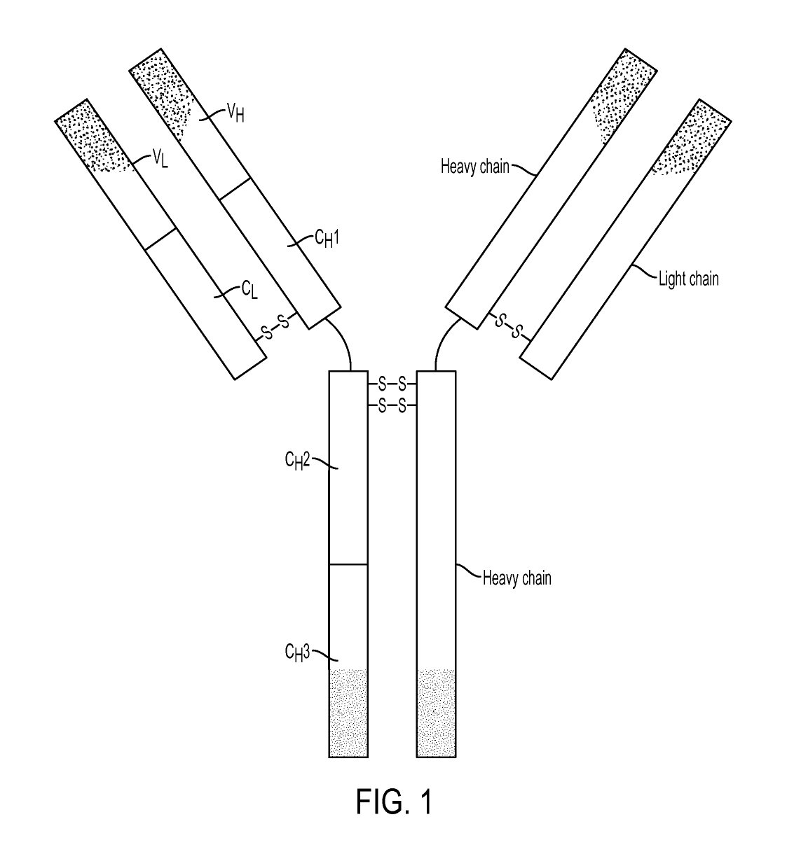

The basic structure of an IgG antibody is illustrated in FIG. 1. As shown in FIG. 1, an IgG antibody consists of two identical light polypeptide chains and two identical heavy polypeptide chains linked together by disulphide bonds. The first domain located at the amino terminus of each chain is variable in amino acid sequence, providing antibody binding specificities found in each individual antibody. These are known as variable heavy (VH) and variable light (VL) regions. The other domains of each chain are relatively invariant in amino acid sequence and are known as constant heavy (CH) and constant light (CL) regions. As shown in FIG. 1, for an IgG antibody, the light chain includes one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies described herein can include, for example, monoclonal antibodies, polyclonal antibodies (e.g., IVIG), multispecific antibodies, human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA and IgY), class (e.g., IgG1, IgG2, IgG3, IgG4, IgA1 and IgA2) or subclass.

The term “Fc fragment”, as used herein, refers to one or more fragments of an Fc region that retains an Fc function and/or activity described herein, such as binding to an Fc receptor. Examples of such fragments include fragments that include an N-linked glycosylation site of an Fc region (e.g., an Asn297 of an IgG heavy chain or homologous sites of other antibody isotypes), such as a CH2 domain. The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). These antibody fragments can be obtained using conventional techniques known to those with skill in the art, and fragments can be screened for utility in the same manner as are intact antibodies.

Reference glycoproteins described herein (e.g., reference antibodies) or fragments thereof can be produced by any method known in the art for synthesizing glycoproteins (e.g., antibodies) (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

Additional reference antibodies described herein are bispecific antibodies and multivalent antibodies, as described in, e.g., Segal et al., J. Immunol. Methods 248:1-6 (2001); and Tutt et al., J. Immunol. 147: 60 (1991).

### C. Amino Acid Modifications of the Fc Region

The amino acid sequence of a glycoprotein described herein can be modified to produce an Fc region variant, such as an Fc region having at least one (e.g., 1, 2, 3, 4, 5, 6, 7, 8, 9, 10, or more) addition, substitution, or deletion of an amino acid residue relative to a reference glycoprotein. Amino acid residue(s) to be modified can be one or more amino acid residue(s) involved in or proximate to an interaction of an Fc region and a glycan, an Fc region and an FcγR, and/or involved in an effector function described herein. For example, crystal structures for Fc dimers with glycans bound to FcγRIII are known (see, e.g., Mizushima et al., Genes to Cells 16:1071-1080 (2011); Ferrara et al., PNAS 108:12669-12674 (2011)). Accordingly, one or more amino acids of the Fc region near or proximal to a bound glycan (e.g., an Fc region amino acid putatively involved in hydrogen bonding and/or Van Der Waals forces with a glycan) can be modified.

Specific, nonlimiting amino acid residues that can be modified include, e.g., F241, F243, K246, T260, Y296, S298, and R301 (Kabat numbering) of an IgG1 immunoglobulin heavy chain, or corresponding amino acid residues of other immunoglobulins. These amino acid residues can be substituted with any amino acid or amino acid analog. For example, substitutions at the recited positions can be made with any naturally-occurring amino acid (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine). In particular instances, an amino acid residue is substituted with alanine.

Glycoproteins described herein can include additional modifications of Fc regions. For example, binding sites on human and murine antibodies for FcγR have been mapped to the “lower hinge region” consisting of residues 233-239 (EU index numbering as in Kabat et al., Sequences of Proteins of Immunological Interest, 5th Ed. Public Health Service, National Institutes of Health, Bethesda, Md. (1991); see Woof et al., Molec. Immunol. 23:319-330 (1986); Duncan et al., Nature 332:563 (1988)). Accordingly, an Fc region variant can include a modification of one or more of amino acids 233-239. Other amino acids that can be modified include G316-K338; K274-R301; and Y407-R416 (Shields et al., J. Biol. Chem. 9:6591-6604 (2001)).

Additionally, a number of different Fc region amino acids that may comprise a binding site for C1q have been identified. These include residues 231-238, 318, 320, 322, and 331 (Kabat numbering) (see, e.g., U.S. Pat. No. 6,194,551; WO 99/51642; Idusogie et al., J. Immunol. 164:4178-4184 (2000). Thus, an Fc region variant can include a modification of one or more of these amino acids (e.g., a modification of 1, 2, 3, 4, 5, 6, 7, 8, 9, 10, or more of these amino acids).

Glycoproteins having one or more amino acid residue modifications described herein can be produced according to molecular biology and recombinant gene expression methods known in the art and described herein.

### D. Glycan Modifications

IgG1 glycosylation typically includes complex type N-glycans with variable galactosylation and low levels of sialylation, as well as low levels of high mannose and hybrid glycans. Therapeutic preparations of glycoproteins described herein can be engineered to include particular levels of glycoproteins having particular glycans. In some embodiments, glycoproteins described herein are transfected and expressed in a host cell, wherein endogenous cellular glycosylation machinery of the host cell produces a glycoprotein having particular glycans.

In some embodiments, glycoproteins described herein are transfected and expressed in a host cell engineered to express one or more exogenous glycosylation enzymes, e.g., one or more glycosyltransferase, e.g., one or more glycosyltransferase described herein, wherein cellular glycosylation machinery of the engineered host cell produces a glycoprotein having particular glycans.

In some embodiments, glycoproteins described herein are transfected and expressed in a host cell engineered to over-express or under-express one or more endogenous glycosylation enzymes, e.g., one or more glycosyltransferase, e.g., one or more glycosyltransferase described herein, wherein cellular glycosylation machinery of the engineered host cell produces a glycoprotein having particular glycans.

In some embodiments, glycoproteins described herein are expressed in a host cell and purified from the host cell, and purified glycoproteins are modified, e.g., enzymatically modified in-vitro with one or more glycosylation enzymes, e.g., one or more glycosyltransferases, e.g., one or more glycosyltransferases disclosed herein, to produce glycoproteins having particular glycans.

### E. Glycoprotein Conjugates

The disclosure includes glycoproteins (or Fc regions or Fc fragments containing one or more N-glycosylation sites thereof) that are conjugated or fused to one or more heterologous moieties and that have different affinities for FcγRs and/or different levels of effector functions as compared with a reference glycoprotein conjugate. Heterologous moieties include, but are not limited to, peptides, polypeptides, proteins, fusion proteins, nucleic acid molecules, small molecules, mimetic agents, synthetic drugs, inorganic molecules, and organic molecules. In some instances, a glycoprotein conjugate is a fusion protein that comprises a peptide, polypeptide, protein scaffold, scFv, dsFv, diabody, Tandab, or an antibody mimetic fused to an Fc region, such as a glycosylated Fc region. A fusion protein can include a linker region connecting an Fc region to a heterologous moiety (see, e.g., Hallewell et al. (1989), J. Biol. Chem. 264, 5260-5268; Alfthan et al. (1995), Protein Eng. 8, 725-731; Robinson & Sauer (1996)).

Exemplary, nonlimiting reference glycoprotein conjugates include abatacept (Orencia®, Bristol-Myers Squibb), alefacept (Amevive®, Astellas Pharma), denileukin diftitox (Ontak®, Eisai), etanercept (Enbrel®, Amgen-Pfizer), and rilonacept (Arcalyst®, Regeneron Pharmaceuticals).

In some instances, a glycoprotein conjugate includes an Fc region (or an Fc fragment containing one or more N-glycosylations site thereof) conjugated to a heterologous polypeptide of at least 10, at least 20, at least 30, at least 40, at least 50, at least 60, at least 70, at least 80, at least 90 or at least 100 amino acids.

In some instances, a glycoprotein conjugate includes an Fc region (or an Fc fragment containing one or more N-glycosylation sites thereof) conjugated to marker sequences, such as a peptide to facilitate purification. A particular marker amino acid sequence is a hexa-histidine peptide, such as the tag provided in a pQE vector (QIAGEN, Inc., 9259 Eton Avenue, Chatsworth, Calif., 91311). Other peptide tags useful for purification include, but are not limited to, the hemagglutinin “HA” tag, which corresponds to an epitope derived from the influenza hemagglutinin protein (Wilson et al., 1984, Cell 37:767) and the “Flag” tag.

In other instances, a glycoprotein conjugate includes an Fc region (or Fc fragment containing one or more N-glycosylation sites thereof) conjugated to a diagnostic or detectable agent. Such fusion proteins can be useful for monitoring or prognosing development or progression of disease or disorder as part of a clinical testing procedure, such as determining efficacy of a particular therapy. Such diagnosis and detection can be accomplished by coupling a glycoprotein to detectable substances including, but not limited to, various enzymes, such as but not limited to horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as but not limited to iodine (¹³¹I, ¹²⁵I, ¹²³I), carbon (¹⁴C), sulfur (³⁵S), tritium (³H), indium (¹¹⁵In, ¹¹³In, ¹¹²In, ¹¹¹In), technetium (⁹⁹Tc), thallium (²⁰¹Ti), gallium (⁶⁸Ga, ⁶⁷Ga), palladium ¹⁰³Pd) molybdenum (⁹⁹Mo), xenon (¹³³Xe), fluorine (¹⁸F), ¹⁵³Sm, ¹⁷⁷Lu, ¹⁵³Gd, ¹⁵⁹Gd, ¹⁴⁹Pm, ¹⁴⁰La, ¹⁶⁹Yb, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ⁵¹Cr, ⁵⁴Mn, ⁷⁵Se, ¹¹³Sn, and ¹¹⁷Sn; positron emitting metals using various positron emission tomographies, non-radioactive paramagnetic metal ions, and molecules that are radiolabelled or conjugated to specific radioisotopes.

Techniques for conjugating therapeutic moieties to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56. (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987)).

### F. Glycan Evaluation

In some embodiments, glycans of glycoproteins are analyzed by any available suitable method. In some instances, glycan structure and composition as described herein are analyzed, for example, by one or more, enzymatic, chromatographic, mass spectrometry (MS), chromatographic followed by MS, electrophoretic methods, electrophoretic methods followed by MS, nuclear magnetic resonance (NMR) methods, and combinations thereof. Exemplary enzymatic methods include contacting a glycoprotein preparation with one or more enzymes under conditions and for a time sufficient to release one or more glycan(s) (e.g., one or more exposed glycan(s)). In some instances, the one or more enzymes include(s) PNGase F. Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some instances, techniques described herein may be combined with one or more other technologies for the detection, analysis, and or isolation of glycans or glycoproteins. For example, in certain instances, glycans are analyzed in accordance with the present disclosure using one or more available methods (to give but a few examples, see Anumula, Anal. Biochem., 350(1):1, 2006; Klein et al., Anal. Biochem., 179:162, 1989; and/or Townsend, R. R. Carbohydrate Analysis” High Performance Liquid Chromatography and Capillary Electrophoresis., Ed. Z. El Rassi, pp 181-209, 1995; WO2008/128216; WO2008/128220; WO2008/128218; WO2008/130926; WO2008/128225; WO2008/130924; WO2008/128221; WO2008/128228; WO2008/128227; WO2008/128230; WO2008/128219; WO2008/128222; WO2010/071817; WO2010/071824; WO2010/085251; WO2011/069056; and WO2011/127322, each of which is incorporated herein by reference in its entirety). For example, in some instances, glycans are characterized using one or more of chromatographic methods, electrophoretic methods, nuclear magnetic resonance methods, and combinations thereof. In some instances, methods for evaluating one or more target protein specific parameters, e.g., in a glycoprotein preparation, e.g., one or more of the parameters disclosed herein, can be performed by one or more of following methods.

In some instances, methods for evaluating one or more target protein specific parameters, e.g., in a glycoprotein preparation, e.g., one or more of the parameters disclosed herein, can be performed by one or more of following methods.

TABLE 1
Exemplary methods of evaluating parameters:

| Method(s) | Relevant literature | Parameter |
|---|---|---|
| C18 UPLC Mass Spec.* | Chen and Flynn, Anal. Biochem., | Glycan(s) |
| | 370: 147-161 (2007) | (e.g., N-linked glycan, exposed |
| | Chen and Flynn, J. Am. Soc. Mass | N-linked glycan, glycan |
| | Spectrom., 20: 1821-1833 (2009) | detection, glycan identification, |
| | | and characterization; site |
| | | specific glycation; glycoform |
| | | detection (e.g., parameters 1-7); |
| | | percent glycosylation; and/or |
| | | aglycosyl) |
| Peptide LC-MS | Dick et al., Biotechnol. Bioeng., | C-terminal lysine |
| (reducing/non-reducing) | 100: 1132-1143 (2008) | |
| | Yan et al., J. Chrom. A., 1164: 153- | |
| | 161 (2007) | |
| | Chelius et al., Anal. Chem., | |
| | 78: 2370-2376 (2006) | |
| | Miller et al., J. Pharm. Sci., | |
| | 100: 2543-2550 (2011) | |
| LC-MS (reducing/non- | Dick et al., Biotechnol. Bioeng., | |
| reducing/alkylated) | 100: 1132-1143 (2008) | |
| | Goetze et al., Glycobiol., 21: 949-959 | |
| | (2011) | |
| Weak cation exchange | Dick et al., Biotechnol. Bioeng., | |
| (WCX) chromatography | 100: 1132-1143 (2008) | |
| LC-MS (reducing/non- | Dick et al., Biotechnol. Bioeng., | |
| reducing/alkylated) | 100: 1132-1143 (2008) | |
| | Goetze et al., Glycobiol., 21: 949-959 | |
| | (2011) | |
| PeptideLC- | Yan et al., J. Chrom. A., 1164: 153- | N-terminal pyroglu |
| MS (reducing/non- | 161 (2007) | |
| reducing) | Chelius et al., Anal. Chem., | |
| | 78: 2370-2376 (2006) | |
| | Miller et al., J. Pharm. Sci., | |
| | 100: 2543-2550 (2011) | |
| Peptide LC-MS | Yan et al., J. Chrom. A., 1164: 153- | Methionine oxidation |
| (reducing/non-reducing) | 161 (2007); | |
| | Xie et al., mAbs, 2: 379-394 (2010) | |
| Peptide LC-MS | Miller et al., J. Pharm. Sci., | Site specific glycation |
| (reducing/non-reducing) | 100: 2543-2550 (2011) | |
| Peptide LC-MS | Wang et al., Anal. Chem., 83: 3133- | Free cysteine |
| (reducing/non-reducing) | 3140 (2011); | |
| | Chumsae et al., Anal. Chem., | |
| | 81: 6449-6457 (2009) | |
| Bioanalyzer | Forrer et al., Anal. Biochem., | Glycan (e.g., N-linked glycan, |
| (reducing/non- | 334: 81-88 (2004) | exposed N-linked glycan) |
| reducing)* | | (including, for example, glycan |
| | | detection, identification, and |
| | | characterization; site specific |
| | | glycation; glycoform detection; |
| | | percent glycosylation; and/or |
| | | aglycosyl) |
| LC-MS (reducing/non- | Dick et al., Biotechnol. Bioeng., | Glycan (e.g., N-linked glycan, |
| reducing/alkylated)* | 100: 1132-1143 (2008) | exposed N-linked glycan) |
| *Methods include | Goetze et al., Glycobiol., 21: 949-959 | (including, for example, glycan |
| removal (e.g., | (2011) | detection, identification, and |
| enzymatic, chemical, | Xie et al., mAbs, 2: 379-394 (2010) | characterization; site specific |
| and physical) of | | glycation; glycoform detection; |
| glycans | | percent glycosylation; and/or |
| | | aglycoosyl) |
| Bioanalyzer | Forrer et al., Anal. Biochem., | Light chain:Heavy chain |
| (reducing/non-reducing) | 334: 81-88 (2004) | |
| Peptide LC-MS | Yan et al., J. Chrom. A., 1164: 153- | Non-glycosylation-related |
| (reducing/non-reducing) | 161 (2007) | peptide modifications |
| | Chelius et al., Anal. Chem., | (including, for example, |
| | 78: 2370-2376 (2006) | sequence analysis and |
| | Miller et al., J. Pharm. Sci., | identification of sequence |
| | 100: 2543-2550 (2011) | variants; |
| | | oxidation; succinimide; |
| | | aspartic acid; and/or site- |
| | | specific aspartic acid) |
| Weak cation exchange | Dick et al., Biotechnol. Bioeng., | Isoforms (including, for |
| (WCX) chromatography | 100: 1132-1143 (2008) | example, charge variants |
| | | (acidic variants and basic |
| | | variants); and/or deamidated |
| | | variants) |
| Anion-exchange | Ahn et al., J. Chrom. B, 878: 403-408 | Sialylated glycan |
| chromatography | (2010) | |
| Anion-exchange | Ahn et al., J. Chrom. B, 878: 403-408 | Sulfated glycan |
| chromatography | (2010) | |
| 1,2-diamino-4,5- | Hokke et al., FEBS Lett., 275: 9-14 | Sialic acid |
| methylenedioxybenzene | (1990) | |
| (DMB) labeling method | | |
| LC-MS | Johnson et al., Anal. Biochem., | C-terminal amidation |
| | 360: 75-83 (2007) | |
| LC-MS | Johnson et al., Anal. Biochem., | N-terminal fragmentation |
| | 360: 75-83 (2007) | |
| Circular dichroism | Harn et al., Current Trends in | Secondary structure (including, |
| spectroscopy | Monoclonal Antibody Development | for example, alpha helix |
| | and Manufacturing, S. J. Shire et al., | content and/or beta sheet |
| | eds, 229-246 (2010) | content) |
| Intrinsic and/or ANS | Harn et al., Current Trends in | Tertiary structure (including, |
| dye fluorescence | Monoclonal Antibody Development | for example, extent of protein |
| | and Manufacturing, S. J. Shire et al., | folding) |
| | eds, 229-246 (2010) | |
| Hydrogen-deuterium | Houde et al., Anal. Chem., 81: 2644- | Tertiary structure and dynamics |
| exchange-MS | 2651 (2009) | (including, for example, |
| | | accessibility f amide protons to |
| | | solvent water) |
| Size-exclusion | Carpenter et al., J. Pharm. Sci., | Extent of aggregation |
| chromatography | 99: 2200-2208 (2010) | |
| Analytical | Pekar and Sukumar, Anal. Biochem., | |
| ultracentrifugation | 367: 225-237 (2007) | |

The literature recited above are hereby incorporated by reference in their entirety or, in the alternative, to the extent that they pertain to one or more of the methods for determining a parameter described herein.

### II. FcγReceptors and Fc Effector Functions

Fc regions of antibodies interact with cellular receptors to mediate antibody-dependent effector functions. For example, in the case of IgGs, different classes of FcγR mediate various cellular responses, such as phagocytosis by macrophages, antibody-dependent cell-mediated cytotoxicity by NK cells, and degranulation of mast cells. Each FcγR displays different binding affinities and IgG subclass specificities.

Effector functions mediated by an antibody Fc region can be divided into two categories. The first type are effector functions that operate after the binding of antibody to an antigen. Such effector functions are mediated by cells of the immune system and include, for example, antibody-dependent cell-mediated cytotoxicity (ADCC) and complement dependent cytotoxicity (CDC) (see, e.g., Daeron, Ann. Rev. Immunol. 15:203-234 (1997); Ward et al., Therapeutic Immunol. 2:77-94 (1995); and Ravetch et al., Ann. Rev. Immunol. 9:457-492 (1991)). The second type are effector functions that operate independently of antigen binding. These include functions that affect half-life, clearance, and the ability to be transferred across cellular barriers by transcytosis (see, e.g., Ward and Ghetie, Therapeutic Immunology 2:77-94 (1995)). Glycoproteins described herein that include an Fc region (or an effector-mediated portion thereof), such as antibodies, antibody fragments that include an Fc region, or glycoprotein conjugates that include an Fc region, can be engineered using methods described herein to exhibit particular levels of one or both of these two classes of effector functions.

### A. Effector Functions Mediated by Fc Receptors

### 1. Types of Fc Receptors

Several effector functions are mediated by Fc receptors (FcRs), which bind the Fc region of an antibody. It has been found that the affinity of a preparation of Fc-containing glycoproteins for an FcR comprising particular glycans can be modified by engineering the preparation to include glycoproteins having particular glycans. Accordingly, methods described herein can be used to modify one or more activity of a therapeutic glycoprotein.

FcRs are defined by their specificity for immunoglobulin isotypes; Fc receptors for IgG antibodies are referred to as FcγR, for IgE as FcεR, for IgA as FcαR and so on. Four subclasses of FcγR have been identified: FcγRI (CD64), FcγRII (CD32), FcγRIII (CD16), and FcγRIV (see, e.g., Nimmerjahn et al., Immunity 24:19-28 (2006)). Because each FcγR subclass is encoded by two or three genes, and alternative RNA spicing leads to multiple transcripts, a broad diversity in FcγR isoforms exists. The three genes encoding the FcγRI subclass (FcγRIA, FcγRIB and FcγRIC) are clustered in region 1q21.1 of the long arm of chromosome 1; the genes encoding FcγRII isoforms (FcγRIIA, FcγRIIB and FcγRIIC) and the two genes encoding FcγRIII (FcγRIIIA and FcγRIIIB) are all clustered in region 1q22. These different FcR subtypes are expressed on different cell types (see Ravetch et al., Ann. Rev. Immunol. 9:457-492 (1991)). For example, in humans, FcγRIIIB is found only on neutrophils, whereas FcγRIIIA is found on macrophages, monocytes, natural killer (NK) cells, and a subpopulation of T-cells.

Structurally, FcγRs are all members of the immunoglobulin superfamily, having an IgG-binding α-chain with an extracellular portion comprised of either two (FcγRI and FcγRIII) or three (FcγRI) Ig-like domains. In addition, FcγRI and FcγRIII have accessory protein chains (γ, ζ) associated with the α-chain, which function in signal transduction. Receptors are also distinguished by their affinity for IgG. FcγRI exhibits a high affinity for IgG, Kₐ=10⁸-10⁹ M⁻¹ (Ravetch et al., Ann. Rev. Immunol. 19:275-290 (2001)) and can bind monomeric IgG. In contrast FcγRII and FcγRIII show a relatively weaker affinity for monomeric IgG Kₐ≦10⁷ M⁻¹ (Ravetch et al., Ann. Rev. Immunol. 19:275-290 (2001)), and hence only interact effectively with multimeric immune complexes. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (see, e.g., Daeron, Ann. Rev. Immunol. 15:203-234 (1997)). NK-cells carry only FcγRIIIA, and binding of antibodies to FcγRIIIA leads to ADCC activity by NK cells.

Allelic variants of several human FcγRs are known to exhibit differences in binding of human and murine IgG, and a number of association studies have correlated clinical outcomes with the presence of specific allelic forms (see Lehrnbecher et al., Blood 94:4220-4232 (1999)). Accordingly, glycoproteins described herein can have different levels of binding to these allelic variants as compared with a reference glycoprotein and can be used as therapeutics for such conditions.

Another type of Fc receptor is the neonatal Fc receptor (FcRn). FcRn is structurally similar to major histocompatibility complex (MHC) and consists of an α-chain noncovalently bound to β2-microglobulin. FcRn has been proposed to regulate homeostasis of IgG in blood as well as possibly control transcytosis across tissues (Ghetie et al., Ann. Rev. Immunol. 18:739-766 (2000)).

Human FcγRIIIa bears five N-linked glycans bound to the asparagine residues at positions 38, 45, 74, 162, and 169 (Ravetch et al., J. Exp. Med. 170:481-497 (1989)).

### 2. Cellular Expression of Fc Receptors

Expression of FcRs varies in different immune cells (see Table 2). It has been found that glycans N-linked to an Fc region of a glycoprotein affect binding of the glycoprotein to FcRs having particular glycans, thereby modulating its effects on different cell types.

TABLE 2
FcγR cellular distribution and effector function

| | Principal | | | |
|---|---|---|---|---|
| Receptor | antibody | Affinity for | | Effect following |
| name | ligand | ligand | Cell distribution | binding to antibody |
| FcγRI (CD64) | IgG1 and | High (Kd ~ | Macrophages | Phagocytosis |
| (CD64) | IgG3 | 10⁻⁹ M) | Neutrophils | Cell activation |
| | | | Eosinophils | Activation of |
| | | | | respiratory burst |
| | | | Dendritic cells | Induction of microbe |
| | | | | killing |
| FcγRIIA | IgG | Low (Kd > | Macrophages | Phagocytosis |
| (CD32) | | 10⁻⁷ M) | Neutrophils | Degranulation |
| | | | Eosinophils | (eosinophils) |
| | | | Platelets | |
| | | | Langerhans cells | |
| FcγRIIB1 | IgG | Low (Kd > | B Cells | No phagocytosis |
| (CD32) | | 10⁻⁷ M) | Mast cells | Inhibition of cell |
| | | | | activity |
| FcγRIIB2 | IgG | Low (Kd > | Macrophages | Phagocytosis |
| (CD32) | | 10⁻⁷ M) | Neutrophils | Inhibition of cell |
| | | | Eosinophils | activity |
| FcγRIIIA | IgG | Low (Kd > | NK cells | Induction of antibody- |
| (CD16a) | | 10⁻⁶ M) | Macrophages | dependent cell-mediated |
| | | | (certain tissues) | cytotoxicity (ADCC) |
| | | | | Induction of cytokine |
| | | | | release by macrophages |
| FcγRIIIB | IgG | Low (Kd > | Eosinophils | Induction of microbe |
| (CD16b) | | 10⁻⁶ M) | Macrophages | killing |
| | | | Neutrophils | |
| | | | Mast cells | |
| | | | Follicular | |
| | | | dendritic cells | |
| FcγRIV | IgG2 | Intermediate | Neutrophils | Activation of cell |
| | | | Monocytes | activity |
| | | | Macrophages | |
| | | | Dendritic cells | |
| FcRn | IgG | | Monocytes | Transfers IgG from a |
| | | | Macrophages | mother to fetus through |
| | | | Dendrite cells | the placenta |
| | | | Epithelial cells | Transfers IgG from a |
| | | | Endothelial cells | mother to infant in milk |
| | | | Hepatocytes | Protects IgG from |
| | | | | degradation |

The 72 kDa extracellular glycoprotein FcγRI is mainly expressed on myeloid cells such as monocytes, macrophages CD4+ progenitor cells and may elicit ADCC, endocytosis, and phagocytosis responses (Siberil et al., 2006, J Immunol Lett 106:111-118). The 40 kDa FcγRII group of receptors (A, B and C isoforms) exhibit extracellular domains but do not contain active signal transduction domains. FcγRIIA is mainly expressed on monocytes, macrophages, neutrophils, and platelets, whereas FcγRIIC receptor has only been identified on NK cells. These two receptors have been shown to initiate ADCC, endocytosis, phagocytosis and inflammatory mediator release (Cassel et al., 1993. Mol Immunol 30:451-60). By contrast, FcγRIIB (B1 and B2 types) receptors are expressed on B cells, Mast cells, basophils, monocytes, macrophages and dendritic cells and have been shown to downregulate immune responses triggered by the A and C isoforms.

The 50 kDa FcγRIIIA is expressed on NK cells, monocytes, macrophages and a subset of T lymphocytes, where it activates ADCC, phagocytosis, endocytosis and cytokine release (Gessner et al., 1998, Ann Hematology 76:231-48). The FcγRIIIB isoform is a glycosyl-phosphatidylinositol (GPI) anchored peripheral membrane protein involved in degranulation and production of reactive oxygen intermediates (Salmon et al., 1995 J. Clin. Inves. 95:2877-2885).

### 3. Binding Properties of Engineered Glycoproteins

Glycoproteins of the disclosure (e.g., therapeutic preparations of glycoproteins described herein) can have altered FcR and/or C1q binding properties (e.g., binding specificity, equilibrium dissociation constant (KD), dissociation and association rates (K_{off} and Kₒₙ respectively), binding affinity and/or avidity), relative to a reference glycoprotein (e.g., a reference glycoprotein preparation). One skilled in the art can determine which kinetic parameter is most important for a given application. For example, methods described herein can be used to produce therapeutic preparations of glycoproteins having an altered level of Fc effector activity as compared with a reference glycoprotein preparation, e.g., having a reduced level of binding to one or more activating Fc receptor (e.g., FcγRIIIA) and/or having an enhanced level of binding to an inhibitory Fc receptor (e.g., FcγRIIB) and thus having a reduced level of ADCC activity as compared with a reference glycoprotein preparation. Alternatively, methods described herein can be used to produce a therapeutic preparation of glycoproteins having an increased level of binding to one or more activating Fc receptor (e.g., FcγRIIIA) and/or having a reduced level of binding to an inhibitory Fc receptor (e.g., FcγRIIB) and thus having an increased level of ADCC activity, as compared with a reference glycoprotein preparation. The ratio of binding affinities (e.g., equilibrium dissociation constants (KD) can indicate if ADCC activity is enhanced or decreased. Additionally, methods described herein can be used to produce a therapeutic preparation of glycoproteins having a reduced level of binding to C1q (and having a reduced or no detectable level of CDC activity), or having an increased level of binding to C1q (and having an increased level of CDC activity), as compared with a reference glycoprotein preparation.

Affinities and binding properties of an Fc region for an FcR and/or C1q can be measured by a variety of in vitro assay methods known in the art for determining Fc-FcγR interactions. Nonlimiting examples of such methods include equilibrium methods (e.g., enzyme-linked immunoabsorbent assay (ELISA) or radioimmunoassay (RIA)), kinetics (e.g., surface plasmon resonance, e.g., BIACORE® analysis), indirect binding assays, competitive inhibition assays, fluorescence resonance energy transfer (FRET), gel electrophoresis, and chromatography (e.g., gel filtration). These and other methods can use a label on one or more of the components being examined and/or employ a variety of detection methods including, but not limited to, chromogenic, fluorescent, luminescent, or isotopic labels.

In some instances, a therapeutic preparation of glycoproteins described herein (e.g., comprising a glycoform having a predetermined glycan profile, e.g., a predetermined or target level of high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits reduced binding affinity for one or more Fc receptors (e.g., comprising one or more terminal glycans selected from mannose, N-acetylglucosamine, beta-1,4 galactose, and sialic acid) including, but not limited to FcγRI (CD64) including isoforms FcγRIA, FcγRIB, and FcγRIC; FcγRII (CD32 including isoforms FcγRIIA, FcγRIIB, and FcγRIIC); and FcγRIII (CD16, including isoforms FcγRIIIA and FcγRIIB), relative to a reference glycoprotein preparation described herein (e.g., lacking the predetermined or target level). In certain embodiments, a therapeutic preparation of glycoproteins described herein does not have increased binding to FcγRIIB receptor as compared to a reference glycoprotein preparation described herein.

In other instances, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a glycan comprising a terminal N-acetylglucosamine, and/or a sulfated glycan) exhibits increased binding affinity for one or more Fc receptors (e.g., comprising one or more terminal glycans selected from mannose, N-acetylglucosamine, beta-1,4 galactose, and sialic acid) including, but not limited to FcγRI (CD64) including isoforms FcγRIA, FcγRIB, and FcγRIC; FcγRII (CD32 including isoforms FcγRIIA, FcγRIIB, and FcγRIIC); and FcγRIII (CD16, including isoforms FcγRIIIA and FcγRIIB), relative to a reference glycoprotein preparation described herein (e.g., lacking the predetermined or target level). In certain embodiments, a therapeutic preparation of glycoproteins described herein has increased binding to FcγRIIB receptor as compared to a reference glycoprotein preparation described herein (e.g., lacking the predetermined or target level).

In one embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits decreased binding affinity to an FcγRI (e.g., comprising one or more terminal glycans selected from mannose, N-acetylglucosamine, beta-1,4 galactose, and sialic acid) relative to a reference glycoprotein preparation described herein (e.g., lacking the predetermined or target level). In another embodiment, a therapeutic preparation of glycoproteins described herein exhibits a binding affinity for the FcγRI receptor that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a reference glycoprotein preparation described herein. In another embodiment, a therapeutic preparation of glycoproteins described herein exhibits an affinity for the FcγRI receptor that is at least 90%, at least 80%, at least 70%, at least 60%, at least 50%, at least 40%, at least 30%, at least 20%, at least 10%, or at least 5% less than a reference glycoprotein preparation described herein.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits increased binding affinity to an FcγRI (e.g., comprising one or more terminal glycans selected from mannose, N-acetylglucosamine, beta-1,4 galactose, and sialic acid) relative to a reference glycoprotein preparation described herein (e.g., lacking the predetermined or target level). In another embodiment, a therapeutic preparation of glycoproteins described herein exhibits a binding affinity for the FcγRI receptor that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold higher than a reference glycoprotein preparation described herein. In another embodiment, a therapeutic preparation of glycoproteins described herein exhibits an affinity for the FcγRI receptor that is at least 5%, at least 10%, at least 20%, at least 30%, at least 40%, at least 50%, at least 60%, at least 70%, at least 80%, or at least 90% higher than a reference glycoprotein preparation described herein.

In one embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits decreased affinity for an FcγRIIIA receptor (e.g., comprising one or more terminal glycans selected from mannose, N-acetylglucosamine, beta-1,4 galactose, and sialic acid) relative to a reference glycoprotein preparation described herein (e.g., lacking the predetermined or target level). In another embodiment, a therapeutic preparation of glycoproteins described herein exhibits an affinity for the FcγRIIIA receptor that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a reference glycoprotein preparation described herein. In another embodiment, a therapeutic preparation of glycoproteins described herein exhibits an affinity for the FcγRIIIA receptor that is at least 90%, at least 80%, at least 70%, at least 60%, at least 50%, at least 40%, at least 30%, at least 20%, at least 10%, or at least 5% less than a reference glycoprotein preparation described herein.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits increased affinity for an FcγRIIIA receptor (e.g., comprising one or more terminal glycans selected from mannose, N-acetylglucosamine, beta-1,4 galactose, and sialic acid) relative to a reference glycoprotein preparation described herein (e.g., lacking the predetermined or target level). In another embodiment, a therapeutic preparation of glycoproteins described herein exhibits an affinity for the FcγRIIIA receptor that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than a reference glycoprotein preparation described herein. In another embodiment, a therapeutic preparation of glycoproteins described herein exhibits an affinity for the FcγRIIIA receptor that is at least 90%, at least 80%, at least 70%, at least 60%, at least 50%, at least 40%, at least 30%, at least 20%, at least 10%, or at least 5% greater than a reference glycoprotein preparation described herein.

The F158V allelic variant of the FcγRIIIA receptor has altered binding characteristics to antibodies. In one embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) binds with decreased affinity to an FcγRIIIA (F158V) (e.g., comprising one or more terminal glycans selected from mannose, N-acetylglucosamine, beta-1,4 galactose, and sialic acid) relative to a reference glycoprotein preparation described herein (e.g., lacking the predetermined or target level). In another embodiment, a therapeutic preparation of glycoproteins described herein exhibits an affinity for the FcγRIIIA (F158V) receptor that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of a reference glycoprotein preparation described herein. In another embodiment, a therapeutic preparation of glycoproteins described herein exhibits an affinity for the FcγRIIIA (F158V) receptor that is at least 90%, at least 80%, at least 70%, at least 60%, at least 50%, at least 40%, at least 30%, at least 20%, at least 10%, or at least 5% less than a reference glycoprotein preparation described herein.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) binds with increased affinity to an FcγRIIIA (F158V) (e.g., comprising one or more terminal glycans selected from mannose, N-acetylglucosamine, beta-1,4 galactose, and sialic acid) relative to a reference glycoprotein preparation described herein (e.g., lacking the predetermined or target level). In another embodiment, a therapeutic preparation of glycoproteins described herein exhibits an affinity for the FcγRIIIA (F158V) receptor that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold higher than that of a reference glycoprotein preparation described herein. In another embodiment, a therapeutic preparation of glycoproteins described herein exhibits an affinity for the FcγRIIIA (F158V) receptor that is at least 90%, at least 80%, at least 70%, at least 60%, at least 50%, at least 40%, at least 30%, at least 20%, at least 10%, or at least 5% higher than a reference glycoprotein preparation described herein.

In another embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits an increased affinity for an FcγRIIB receptor (e.g., comprising one or more terminal glycans selected from mannose, N-acetylglucosamine, beta-1,4 galactose, and sialic acid) as compared to a reference glycoprotein preparation described herein (e.g., lacking the predetermined or target level). In another embodiment, a therapeutic preparation of glycoproteins described herein exhibits an affinity for the FcγRIIB receptor that is unchanged or increased by at least at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold than that of a reference glycoprotein preparation described herein. In another embodiment, a therapeutic preparation of glycoproteins described herein exhibits an affinity for the FcγRIIB receptor that is increased by at least 5%, at least 10%, at least 20%, at least 30%, at least 40%, at least 50%, at least 60%, at least 70%, at least 80%, at least 90%, or at least 95% relative to a reference glycoprotein preparation described herein.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits a decreased affinity for an FcγRIIB receptor (e.g., comprising one or more terminal glycans selected from mannose, N-acetylglucosamine, beta-1,4 galactose, and sialic acid) as compared to a reference glycoprotein preparation described herein (e.g., lacking the predetermined or target level). In another embodiment, a therapeutic preparation of glycoproteins described herein exhibits an affinity for the FcγRIIB receptor that is decreased by at least at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold than that of a reference glycoprotein preparation described herein. In another embodiment, a therapeutic preparation of glycoproteins described herein exhibits an affinity for the FcγRIIB receptor that is decreased by at least 5%, at least 10%, at least 20%, at least 30%, at least 40%, at least 50%, at least 60%, at least 70%, at least 80%, at least 90%, or at least 95% relative to a reference glycoprotein preparation described herein.

In another embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits an affinity for an FcγRI, FcγRIIIA, or FcγRIIIA (F158V) receptor (e.g., comprising one or more terminal glycans selected from mannose, N-acetylglucosamine, beta-1,4 galactose, and sialic acid) that is about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain embodiments, a therapeutic preparation of glycoproteins described herein exhibits an affinity for the FcγRI, FcγRIIIA, or FcγRIIIA (F158V) receptor that is greater than about 1 μM, greater than about 5 μM, greater than about 10 μM, greater than about 25 μM, greater than about 50 μM, or greater than about 100 μM.

In another embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits an affinity for an FcγRIIB receptor (e.g., comprising one or more terminal glycans selected from mannose, N-acetylglucosamine, beta-1,4 galactose, and sialic acid) that is about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain embodiments, a therapeutic preparation of glycoproteins described herein exhibits an affinity for the FcγRI, FcγRIIIA, or FcγRIIIA (F158V) receptor that is less than about 100 μM, less than about 50 μM, less than about 10 μM, less than about 5 μM, less than about 2.5 μM, less than about 1 μM, less than about 100 nM, or less than about 10 nM.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits an affinity for an FcγRIIB receptor (e.g., comprising one or more terminal glycans selected from mannose, N-acetylglucosamine, beta-1,4 galactose, and sialic acid) that is between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain embodiments, a therapeutic preparation of glycoproteins described herein exhibits an affinity for the FcγRI, FcγRIIIA, or FcγRIIIA (F158V) receptor that is less than about 100 μM, less than about 50 μM, less than about 10 μM, less than about 5 μM, less than about 2.5 μM, less than about 1 μM, less than about 100 nM, or less than about 10 nM.

### 4. ADCC Activity of Engineered Glycoproteins

Methods described herein can be used to produce therapeutic preparations of glycoproteins that can induce antibody-dependent cell-mediated cytotoxicity (“ADCC”) at an altered level as compared with a reference glycoprotein preparation. ADCC refers to a form of cytotoxicity in which secreted Ig bound to Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” cytotoxic cells and are required for such killing. Lysis of the target cell is extracellular, requires direct cell-to-cell contact, and does not involve complement.

The ability of a glycoprotein to mediate lysis of a target cell by ADCC can be assayed. To assess ADCC activity, a therapeutic preparation of glycoproteins or a reference glycoprotein preparation can be added to target cells in combination with immune effector cells, which can be activated by an antigen antibody complex, resulting in cytolysis of the target cell. Cytolysis can be detected, such as by detecting release of a label (e.g., radioactive substrates, fluorescent dyes or natural intracellular proteins) from lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. ADCC activity can also be assessed in vivo, e.g., in an animal model, such as that disclosed in Clynes et al., 1998, PNAS USA 95:652-656.

In one embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits decreased ADCC activity relative to a reference glycoprotein preparation described herein (e.g., lacking the predetermined or target level). In some embodiments, a therapeutic preparation of glycoproteins described herein exhibits ADCC activity that is at least 1.2 fold, 1.5 fold, 1.8 fold, 2 fold, or at least 3 fold, or at least 5 fold, or at least 10 fold, or at least 50 fold, or at least 100 fold less than that of a reference glycoprotein preparation described herein. In still another embodiment, a therapeutic preparation of glycoproteins described herein exhibits ADCC activity that is reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to a reference glycoprotein preparation described herein. In certain embodiments, a therapeutic preparation of glycoproteins described herein exhibits no detectable ADCC activity.

In other embodiments, a therapeutic preparation of glycoproteins described herein (e.g., comprising a glycoform having predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits increased ADCC activity relative to a reference glycoprotein preparation described herein (e.g., lacking the predetermined or target level). In some embodiments, a therapeutic preparation of glycoproteins described herein exhibits ADCC activity that is at least 1.2 fold, 1.5 fold, 1.8 fold, 2 fold, or at least 3 fold, or at least 5 fold, or at least 10 fold, or at least 50 fold, or at least 100 fold higher than that of a reference glycoprotein preparation described herein. In still another embodiment, a therapeutic preparation of glycoproteins described herein exhibits ADCC activity that is increased by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to a reference glycoprotein preparation described herein.

### B. Effector Functions Mediated by Complement

Another antibody effector function is “complement dependent cytotoxicity”, or “CDC”, which refers to a biochemical event of antibody-mediated target cell destruction by the complement system. The complement system is a complex system of proteins found in normal blood plasma that combines with antibodies to destroy pathogenic bacteria and other foreign cells.

### 1. C1q Binding

C1q and two serine proteases, C1r and C1s, form the complex C1, the first component of the CDC pathway, and Fc binding to C1q mediates CDC (see Ward et al., Therapeutic Immunology 2:77-94 (1995)).

In one embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits decreased affinity to C1q relative to a reference glycoprotein preparation described herein (e.g., lacking the predetermined or target level). In another embodiment, a therapeutic preparation of glycoproteins described herein exhibits an affinity for C1q that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a reference glycoprotein preparation described herein. In another embodiment, a therapeutic preparation of glycoproteins described herein exhibits an affinity for C1q that is at least 90%, at least 80%, at least 70%, at least 60%, at least 50%, at least 40%, at least 30%, at least 20%, at least 10%, or at least 5% less than a reference glycoprotein preparation described herein.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits increased affinity to C1q relative to a reference glycoprotein preparation described herein (e.g., lacking the predetermined or target level). In another embodiment, a therapeutic preparation of glycoproteins described herein exhibits an affinity for C1q that is at least 1.2 fold, 1.5 fold, 1.8 fold, 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold higher than a reference glycoprotein preparation described herein. In another embodiment, a therapeutic preparation of glycoproteins described herein exhibits an affinity for C1q that is at least 90%, at least 80%, at least 70%, at least 60%, at least 50%, at least 40%, at least 30%, at least 20%, at least 10%, or at least 5% higher than a reference glycoprotein preparation described herein.

In another embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits an affinity for C1q that is about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain embodiments, a therapeutic preparation of glycoproteins described herein exhibits an affinity for C1q that is greater than about 1 μM, greater than about 5 μM, greater than about 10 μM, greater than about 25 μM, greater than about 50 μM, or greater than about 100 μM.

### 2. CDC Activity Mediated by Engineered Glycoproteins

In some embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) can exhibit an altered level of complement activation, e.g., relative to a reference glycoprotein preparation described herein (e.g., lacking the predetermined or target level). Any known CDC assay (such as described, e.g., in Gazzano-Santoro et al., 1996, J. Immunol. Methods, 202:163), can be performed to assess complement activation. In one nonlimiting, exemplary method, various concentrations of a therapeutic preparation of glycoproteins or a reference glycoprotein preparation and human complement are diluted with buffer. Cells that express an antigen to which a glycoprotein binds are diluted to a density of about 1×10⁶ cells/mL. Mixtures of a therapeutic preparation of glycoproteins or a reference glycoprotein preparation, diluted human complement, and cells expressing the antigen are added to a flat bottom tissue culture 96 well plate and allowed to incubate for 2 hrs at 37° C. and 5% CO₂ to facilitate complement mediated cell lysis. 50 μL of alamar blue (Accumed International) is then added to each well and incubated overnight at 37° C. Absorbance is measured using a 96-well fluorometer with excitation at 530 nm and emission at 590 nm. Results can be expressed in relative fluorescence units (RFU). Sample concentrations can be computed from a standard curve, and percent activity of the therapeutic preparation of glycoproteins described herein is compared to that of the reference glycoprotein preparation described herein. A difference in percent activity of the therapeutic preparation of glycoproteins described herein compared to that of the reference glycoprotein preparation described herein indicates that the therapeutic preparation of glycoproteins described herein exhibits a modified level of complement activation, e.g., relative to a corresponding reference glycoprotein preparation described herein.

In one embodiment, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits decreased CDC activity relative to a reference glycoprotein preparation described herein (e.g., lacking the predetermined or target level). In another embodiment, a therapeutic preparation of glycoproteins described herein exhibits CDC activity that is at least 1.2 fold, 1.5 fold, 1.8 fold, 2 fold, or at least 3 fold, or at least 5 fold, or at least 10 fold, or at least 50 fold, or at least 100 fold less than that of a reference glycoprotein preparation described herein. In still another embodiment, a therapeutic preparation of glycoproteins described herein exhibits CDC activity that is reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to a reference glycoprotein preparation described herein. In certain embodiments, a therapeutic preparation of glycoproteins described herein exhibits no detectable CDC activity.

In other embodiments, a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) exhibits increased CDC activity relative to a reference glycoprotein preparation described herein (e.g., lacking the predetermined or target level). In another embodiment, a therapeutic preparation of glycoproteins described herein exhibits CDC activity that is at least 1.2 fold, 1.5 fold, 1.8 fold, 2 fold, or at least 3 fold, or at least 5 fold, or at least 10 fold, or at least 50 fold, or at least 100 fold higher than that of a reference glycoprotein preparation described herein. In still another embodiment, a therapeutic preparation of glycoproteins described herein exhibits CDC activity that is increased by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to a reference glycoprotein preparation described herein.

### C. Other Properties of Engineered Glycoproteins

Therapeutic preparations of glycoproteins described herein (e.g., comprising a predetermined or target level of glycoproteins having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) can exhibit altered levels of clearance, half-life, apoptosis, and/or phagocytosis, relative to a reference glycoprotein preparation (e.g., lacking the predetermined or target level). For example, a therapeutic preparation of glycoproteins can have an altered level of binding affinity for FcRn, and thus have an altered level of clearance and/or half-life properties as compared with a reference glycoprotein preparation (see, e.g., D'Acqua et al., J. Immunol. 169:1571-1580 (2002)).

### III. Recombinant Gene Expression

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a gene encoding a polypeptide, such as an antibody or an enzyme described herein, can include construction of an expression vector containing a polynucleotide that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and transfected cells can then cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E. coli and B. subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g. Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For bacterial systems, a number of expression vectors can be used, including, but not limited to, the E. coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 81:355-359). The efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes the gene product in the specific fashion desired. Different host cells have characteristic and specific mechanisms for post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, but are not limited to, CHO, VERY, BHK, HeLa, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a glycoprotein described herein been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a glycoprotein can be fused to heterologous polypeptide sequences to facilitate purification. Glycoproteins having desired sugar chains can be separated with a lectin column by methods known in the art (see, e.g., WO 02/30954).

### IV. Pharmaceutical Compositions and Administration

A glycoprotein described herein (e.g., a therapeutic preparation of glycoproteins described herein, e.g., comprising a predetermined or target level of glycoproteins having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan) can be incorporated into a pharmaceutical composition. Such a pharmaceutical composition is useful as an improved composition for prevention and/or treatment of diseases relative to a reference glycoprotein preparation. Pharmaceutical compositions can be formulated by methods known to those skilled in the art (see, e.g., Remington's Pharmaceutical Sciences pp. 1447-1676 (Alfonso R. Gennaro, ed., 19th ed. 1995)). Pharmaceutical compositions can be administered parenterally in the form of an injectable formulation comprising a sterile solution or suspension in water or another pharmaceutically acceptable liquid. For example, a pharmaceutical composition can be formulated by suitably combining a preparation of glycoproteins with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

A suitable means of administration can be selected based on the age and condition of the subject. A single dose of a pharmaceutical composition containing a therapeutic preparation of glycoproteins can be selected from a range of 0.001 to 1000 mg/kg of body weight. On the other hand, a dose can be selected in the range of 0.001 to 100000 mg/body weight, but the present disclosure is not limited to such ranges. Dose and method of administration can vary depending on the weight, age, condition, and the like of the subject, and can be suitably selected as needed by those skilled in the art.

In some embodiments, a subject is selected for treatment with a preparation of glycoproteins described herein. For example, FcRs from a biological sample (e.g., cells, e.g., PBMC or NK cells) from the subject are characterized and the identities of one or more glycans on the FcRs are determined. Based on the glycans present on the FcRs, the subject is selected for treatment with a therapeutic preparation of glycoproteins described herein (e.g., comprising a glycoform having a predetermined or target level of glycans having a high mannose glycan, a sialylated glycan (e.g., a branched glycan having a sialic acid on one or both of an α1,3 arm and an α1,6 arm (e.g., with a NeuAc-α2,6-Gal terminal linkage)), a galactosylated glycan, a fucosylated glycan, a glycan comprising a terminal N-acetylglucosamine, and/or a sulfated glycan). In some embodiments, the subject is treated with the therapeutic preparation of glycoproteins.

In some instances, the level of binding of a glycoprotein preparation described herein to one or more FcRs described herein can be compared to a target value (e.g., reference standard), e.g., to make a decision regarding the composition of the glycoprotein preparation, e.g., a decision to classify, select, accept or discard, release or withhold, process into a drug product, ship, move to a different location, formulate, label, package, release into commerce, or sell or offer for sale the glycoprotein preparation. In other instances, the decision can be to accept, modify or reject a production parameter or parameters used to make the glycoprotein. Particular, nonlimiting examples of reference standards include a control level (e.g., a level of binding to one or more FcRs or FcR glycoforms by a reference glycoprotein preparation described herein) or a range or value in a product specification or quality criterion (e.g., an FDA label, Physician's Insert, Certificate of Testing, Certificate of Analysis, Master Batch Record) for a pharmaceutical preparation containing the glycoprotein preparation.

In some instances, methods (i.e., evaluation, identification, and production methods) include taking action (e.g., physical action) in response to the methods disclosed herein. For example, a glycoprotein preparation is classified, selected, accepted or discarded, released or withheld, processed into a drug product, shipped, moved to a different location, formulated, labeled, packaged, released into commerce, or sold or offered for sale, depending on whether the preselected or target value is met. In some instances, processing may include formulating (e.g., combining with pharmaceutical excipients), packaging (e.g., in a syringe or vial), labeling, or shipping at least a portion of the glycoprotein preparation. In some instances, processing includes formulating (e.g., combining with pharmaceutical excipients), packaging (e.g., in a syringe or vial), and labeling at least a portion of the preparation as a drug product described herein. Processing can include directing and/or contracting another party to process as described herein.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1

### Affinities of Glycoengineered Fc Molecules for Glycoengineered FcγRIIIa

To map how specific FcR glycoforms interact with specific IgG glycoforms, specific glycoforms of the FcR and the IgG were designed and generated using a combination of glycosidases and glycosyl transferases. This matrix of glycoforms was then used for interaction studies using surface plasmon resonance (SPR) techniques.

An initial screening was performed on soluble recombinant human FcγRIIIa truncated at Gln208 expressed in murine NS0 cells (srhFcγRIIIa, available from R&D Systems, Minneapolis, Minn.). The protein was initially incubated with endoproteinase GluC to ensure C-terminal cleavage. The glycosylation at Asn162 was extremely variable, with more than 12 high abundance glycoforms found. Glycoforms varied in antennarity, and the presence of antennary fucose.

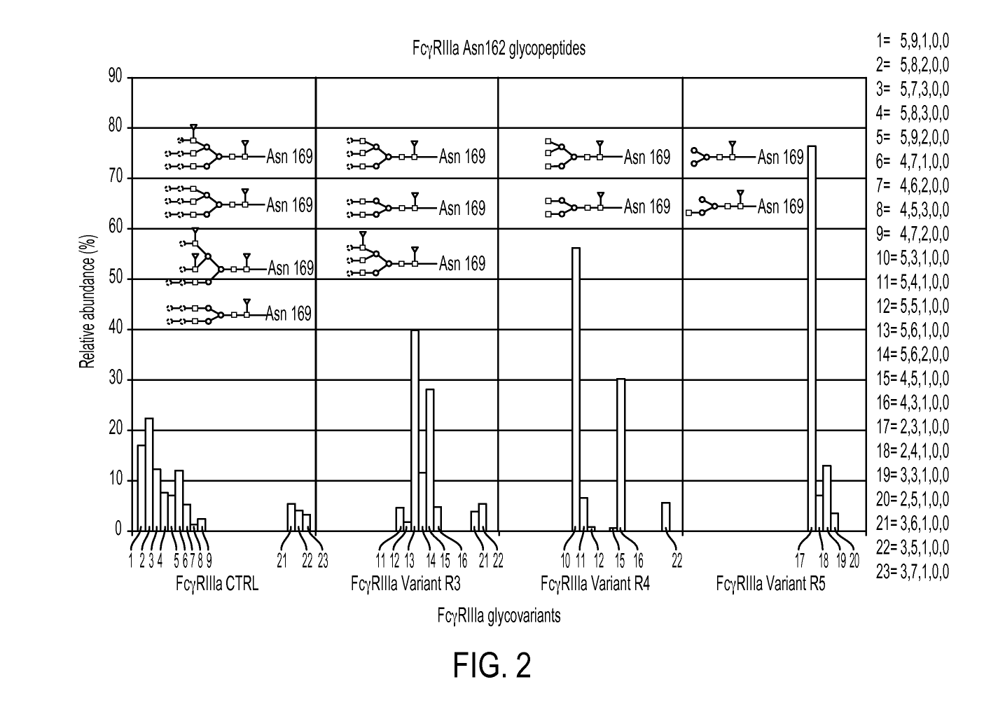

Different glycoforms of srhFcγRIIIa were generated using glycosidic enzymes. FIG. 2 includes the structures of species with relative abundance >10% as determined by LC-MS quantitation of the Asparagine 162 glycopeptides generated by treatment of the protein with a combination of trypsin and endoproteinase GluC.

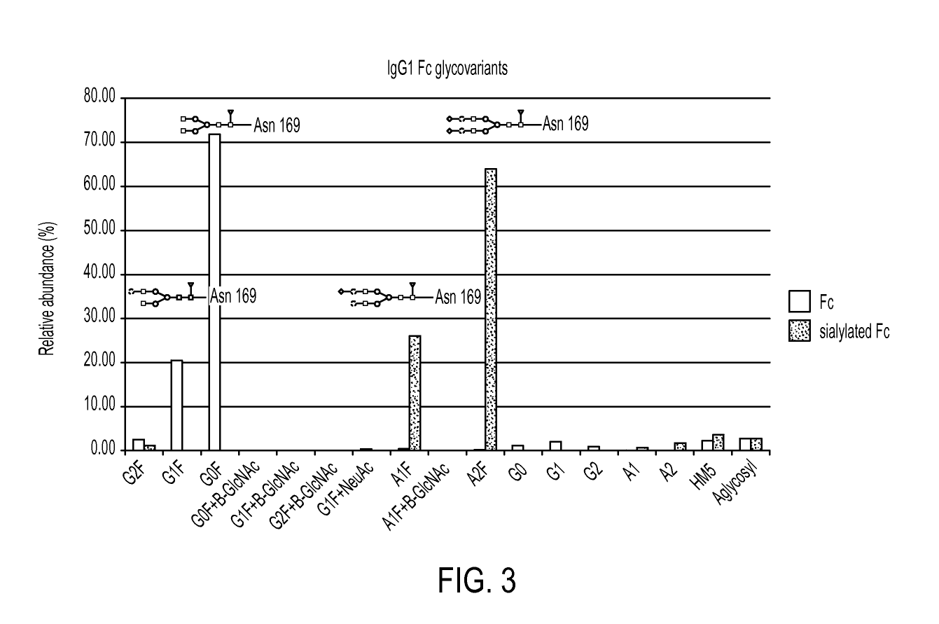

An IgG1 Fc preparation was generated through enzymatic cleavage in the hinge region of a mixture of hIgG isotypes using papain. Sialylated Fc was generated enzymatically by incubating with a combination of galactosyltransferase (B4GalT) and sialylatransferase (ST6Gal) enzymes. The N-linked glycans at Asn297 were analyzed and quantified by LC-MS analysis of the tryptic glycopeptides. As shown in FIG. 3, the IgG1 Fc glycovariants included predominantly neutral glycans as well as sialylated Fc glycovariants having predominantly acidic glycans.

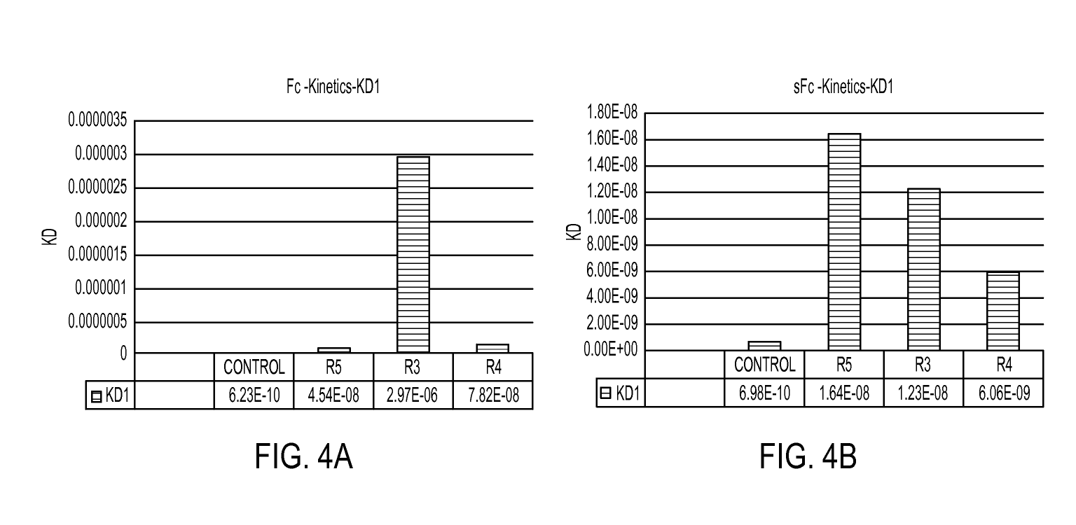

The affinities of the Fc glycoforms for the srhFcγRIIIa glycoforms were determined. As shown in FIG. 4, the affinities for non-sialylated Fc (FIG. 4A) and sialylated Fc (FIG. 4B) for a control receptor were similar. However, the affinity of the Fc glycoforms for the modified receptors was different. Removal of the α-galactose and antennary fucose from the control receptor (resulting in variant “R3” depicted in FIG. 2) resulted in a 4000 fold decrease in affinity for the non-sialylated Fc (FIG. 4A, “R3”) but only a 100 fold decrease in affinity for the sialylated Fc (FIG. 4B, “R3”). Subsequent removal of the β-galactose residues from the receptor (resulting in variant “R4” depicted in FIG. 2) resulted in a 300 fold increase in affinity for the non-sialylated Fc (FIG. 4A, “R4”) but only a 2 fold increase in affinity for the sialylated Fc (FIG. 4B, “R4”). Removal of N-acetylglucosamine from the receptor (resulting in variant “R5” depicted in FIG. 2) did not significantly change the affinity for either non-sialylated Fc (FIG. 4A, “R5”) or sialylated Fc (FIG. 4B, “R5”).

These data demonstrate that the effect of IgG1 glycosylation on FcγRIIIa binding is dependent on the glycosylation of the receptor. Further, the level of binding of Fc and sialylated Fc glycoforms to these FcγRIIIa receptors was surprising, given that a high level of the Fc and sialylated Fc glycoforms included core fucosylation.

### Example 2

### Affinities of Glycoengineered Fc for FcγRIIIa from Donor NK Cells

Human PBMC were isolated from fresh human blood from four donors, and NK cells (expressing FcγRIIIa) were used as effector cells. Raji cells expressing CD20 were used as target cells. Cell killing of Raji cells by NK cells was measured and correlated to the amounts of LDH released from lysed cells. ADCC is primarily mediated by NK cells after binding of an immune complex to CD16 (FcγRIIIa) receptor, which activates downstream cascade leading to a release of perforin and granzyme molecules. Thus, induction of ADCC provides insight into binding to FcγRIIIa.

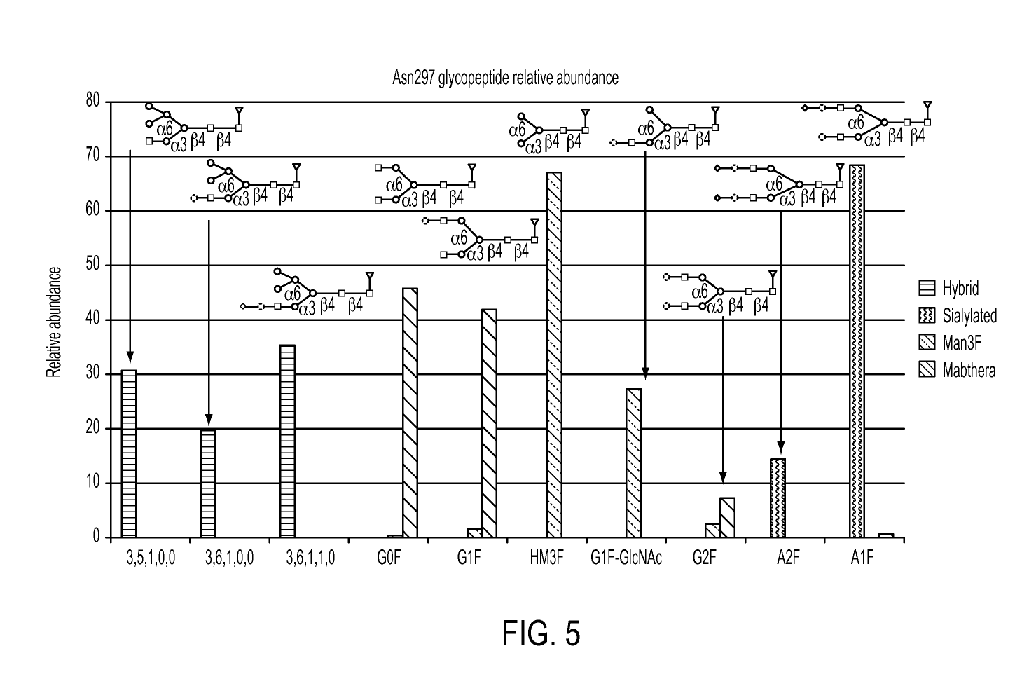

Preparations of rituximab (anti-CD20 antibody) glycoforms were prepared enzymatically, and the glycans from the glycoforms were analyzed by LC-MS analysis of the tryptic digest quantifying the glycopeptides attached at Asn297. Briefly, proteins were denatured and the cysteine residues were reduced and alkylated with iodoacetamide. The protein was then proteolytically digested with a combinantion of trypsin and endoproteinase GluC. The glycans from rituximab glycoform preparations (“hybrid”, “sialylated”, “M3”) and control rituximab (“control”) are depicted in FIG. 5.

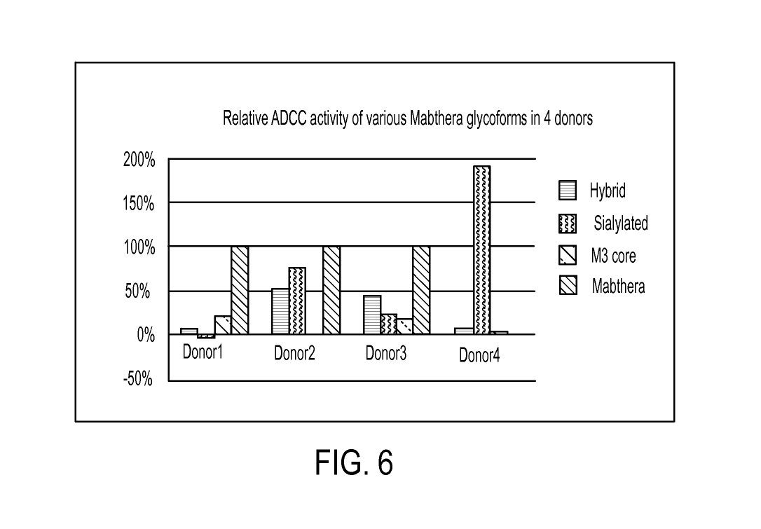

The abilities of the rituximab glycoforms to induce ADCC killing of Raji cells were assayed. As shown in FIG. 6, control rituximab (not modified enzymatically) was essentially inactive in inducing ADCC by NK cells from donor 4, and sialylated glycoforms partially restored ADCC activity. For NK cells from donors 1, 2, and 3, ADCC induced by all rituximab glycoforms was lower than that induced by control rituximab (FIG. 6). Thus, specific Fc glycoforms were able to induce various levels of ADCC by NK cells from different donors.

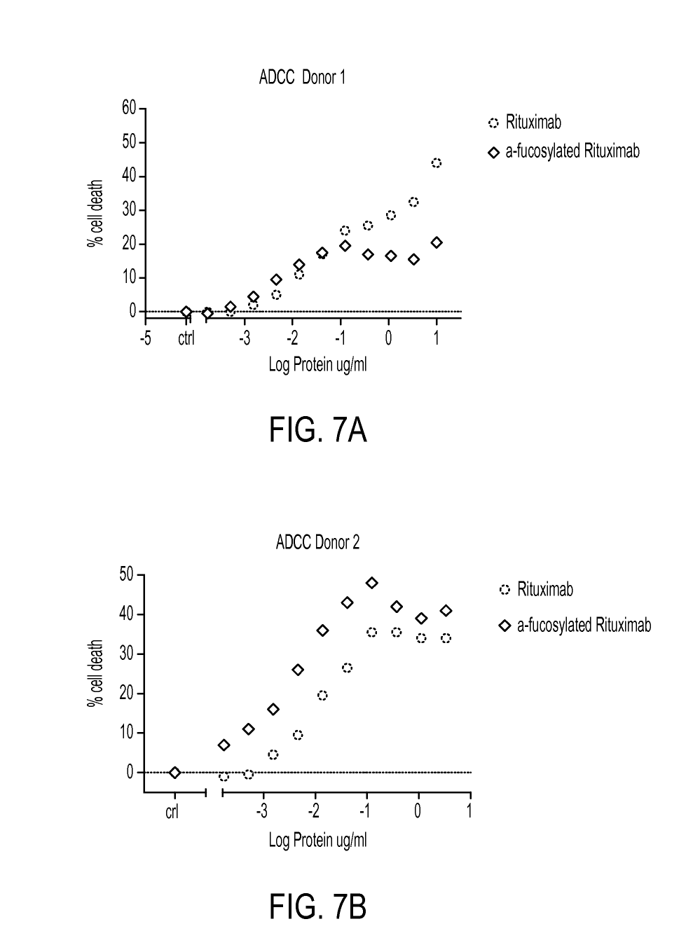

Afucosylated rituximab is known to have improved binding to FcγRIIIa (Kanda et al., Glycobiology 17:104-118 (2006)). Unexpectedly, as shown in FIG. 7A, in donor 1, afucosylated rituximab was less effective than control rituximab in inducing ADCC. In donor 2, hybrid afucosylated rituximab was more effective than afucosylated rituximab or control rituximab in inducing ADCC (data not shown). Further, afucosylated rituximab was more effective than control rituximab in inducing ADCC (FIG. 7B). While not wishing to be bound by theory, it is believed that these differences in ADCC were the result of different Fc receptor glycoforms on NK cells from these different donors.

### Example 3

### Additional Characterization of Affinities of Glycoengineered Fc Molecules for Glycoengineered FcγRIIIa

### Methods

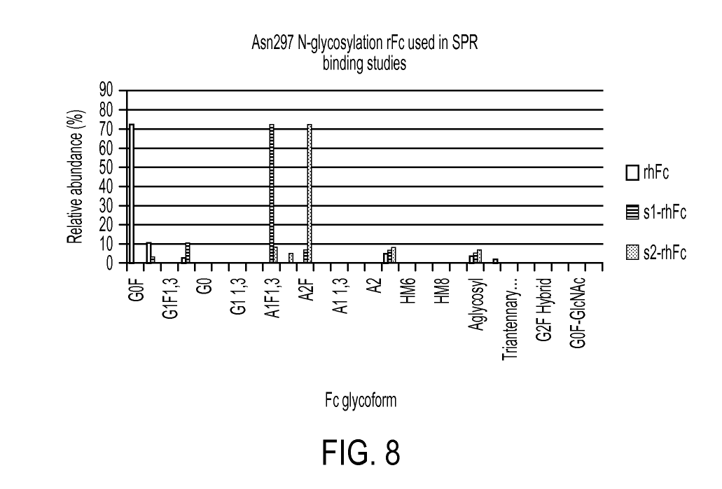

Recombinant human Fc domain of IgG1 (rFc) was expressed in CHO cells. Sialylation was performed by sequential reaction with B4GalT1 and ST6Gal in the presence of UDP-Gal and CMP-NANA as follows, without purification between reactions. The rFc substrate was incubated at 37° C. for 24-48 hours with 50 mM UDP-galactose and 20 mU of bovine milk beta-1,4-galactosyltransferase per mg of substrate. The galactosylated substrate was further incubated at 37° C. with CMP-sialic acid and alpha-2,6-sialyltransferase (ST6). Monosialylated rFc (“s1-rFc”) was generated by galactosylation at an rFc concentration of 30 mg/mL and then incubation for 6 hours with 46 mU of ST6 per mg of rFc and 80 mM CMP-sialic acid. Disialylated rFc (“s2-rFc”) was produced by galactosylation at an rFc concentration of 60 mg/mL followed by incubation for 24 hours with 306 mU of ST6 per mg of rFc and 20 mM CMP-sialic acid, with the addition of another 80 mM CMP-sialic acid 18 hours after adding ST6. Enzyme activity was determined as described in Anumula, Glycobiol. 22:912-917 (2012). The s1-rFc product contained predominantly A1F glycosylation, with the sialic acid on the α1,3 branch, while the s2-rFc product contained predominantly A2F glycosylation, with sialic acid on both the α1,3 and α1,6 branches (FIG. 8).

FcγRIIIa (CD16a) was expressed and purified from CHO, HEK, and NS0 cells. Desialylated FcγRIIIa (CHO deS) was prepared from FcγRIIIa expressed in CHO cells by reacting with sialylidase A for 3 hours at 37° C.

FcγRIIIa samples were digested with GluC and chymotrypsin and analyzed by LC-MS/MS using a reverse phase separation. Glycopeptides were quantified based on the extracted ion area of the z=3 ion for each of the N162 glycopeptides.

The rhFc glycopeptides were generated by digesting rFc samples with trypsin. Quantitation was by LC-MS based on the extracted ion chromatogram for the two most abundant charge states.

Equilibrium binding assays (using Surface Plasmon Resonance “SPR”) were carried out on a ProteOn XPR36 system. Briefly, Fc glycoforms (probes) were immobilized to a GLC sensor surface by direct amine coupling and passed over with receptors in PBS-T0.05% running buffer at 30 μL/min. At least three replicates were measured per receptor. The probe surface was regenerated with an 18 s pulse of 10 mM glycine, pH 1.5 in between analyte injections. Data was analyzed in ProteOn Manager software.

### Results

Receptors from Different Sources Vary in Glycoform

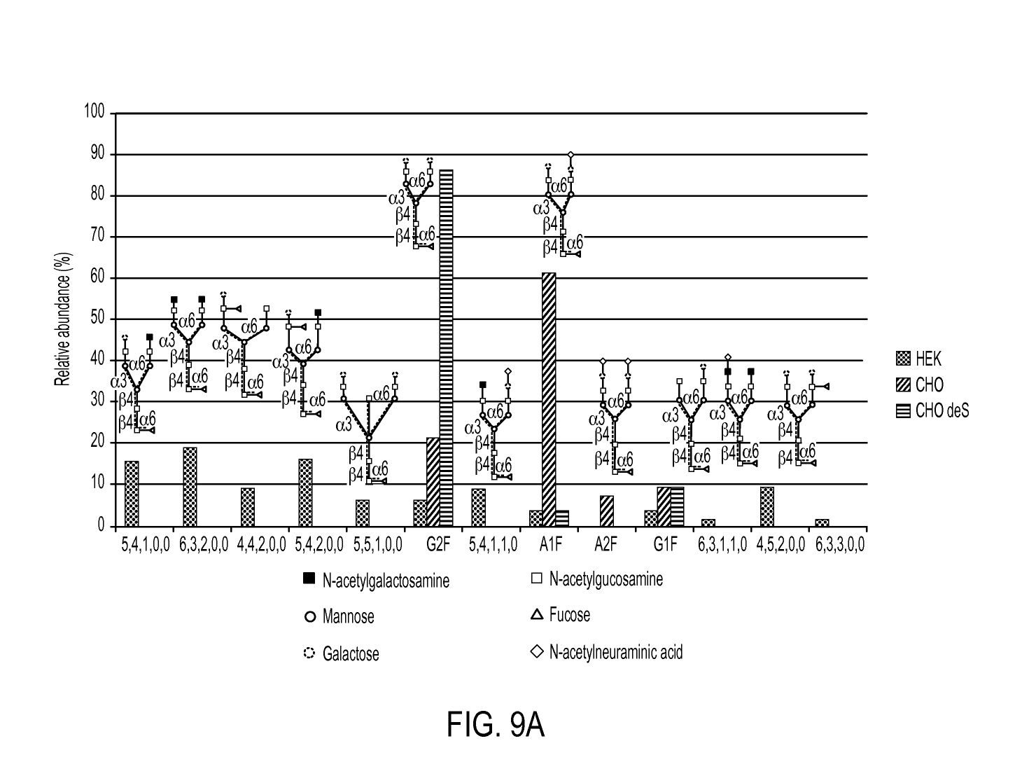

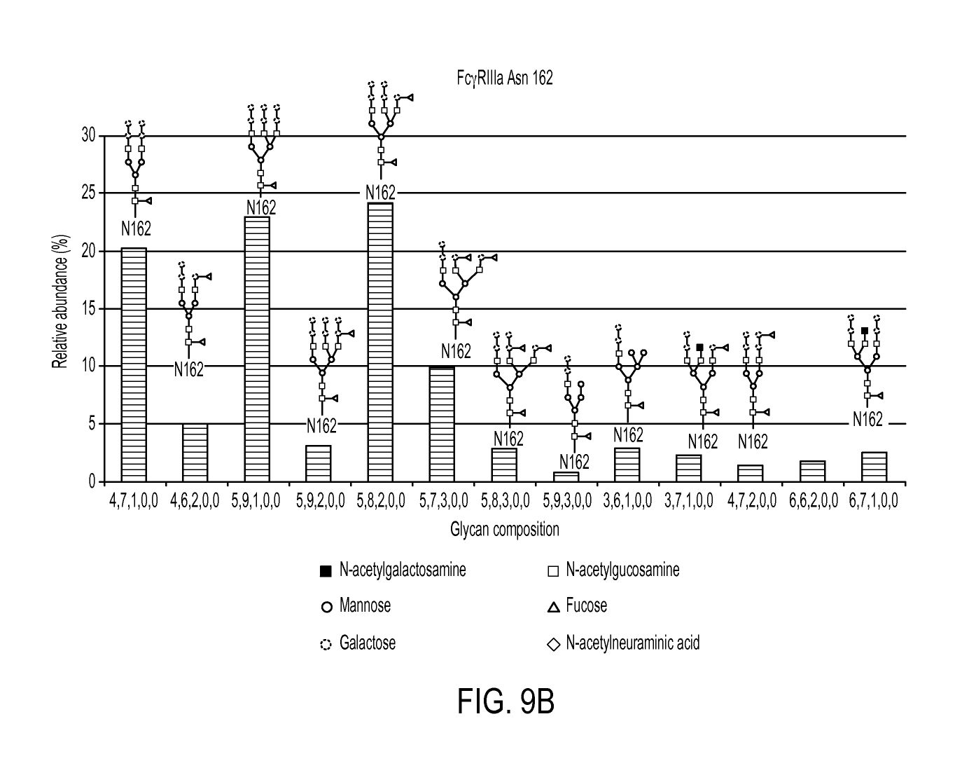

Glycosylation of rhFcγRIIIa varied based on the cell type in which it was expressed. Glycans at N162 from rhFcγRIIIa expressed in HEK cells contained primarily terminal N-acetylgalactosamine with and without antennary fucose (FIG. 9A). Glycosylation at N162 of CHO cell expressed rhFcγRIIIa was characterized by equal levels of terminal sialic acid and terminal galactose with no antennary fucose (FIG. 9A). The N-glycans at N162 in NS0 cells was characterized by terminal α-galactose with high levels of antennary fucose (FIG. 9B).

Receptor Affinity for Fc-Domain of IgG Depends on Receptor Isoform, Receptor Glycoform, and IgG Fc Glycoform

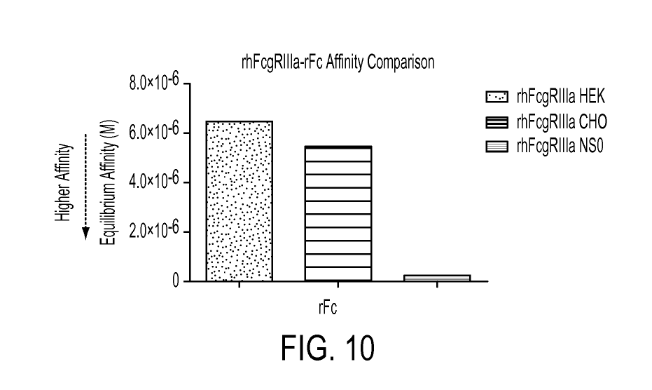

To evaluate the interplay between Fc-receptor and IgG Fc glycosylation on their interaction, the binding of a recombinant Fc with a glycosylation profile typical of a recombinant therapeutic antibody (rFc) to FcγRIIIa with different glycoform distributions was first evaluated using SPR binding studies. The recombinant Fc showed higher affinity for FcγRIIIa expressed in NS0 cells when compared to FcγRIIIa expressed in HEK or CHO cells (FIG. 10). As shown in FIG. 9, the main difference in these Fc receptors was their glycosylation profile. Therefore, the observed differences in the interaction with the rFc may be due to differences in Fc receptor glycosylation patterns.

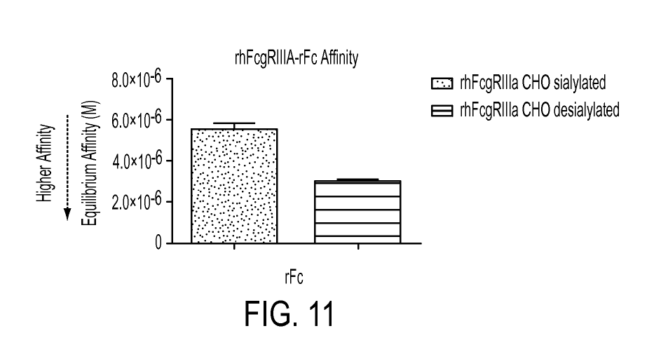

Next, the effect of FcγRIIIa terminal glycans on rFc binding was evaluated by determining the interaction between the rFc and the FcγRIIIa expressed in CHO cells before and after desialylation of the FcγRIIIa receptor. Interestingly, desialylating the same FcγRIIIa receptor expressed in CHO cells caused a small increase in the affinity between the receptor and the recombinant Fc relative to the sialylated (non-desialylated) FcγRIIIa receptor expressed in CHO cells (FIG. 11).

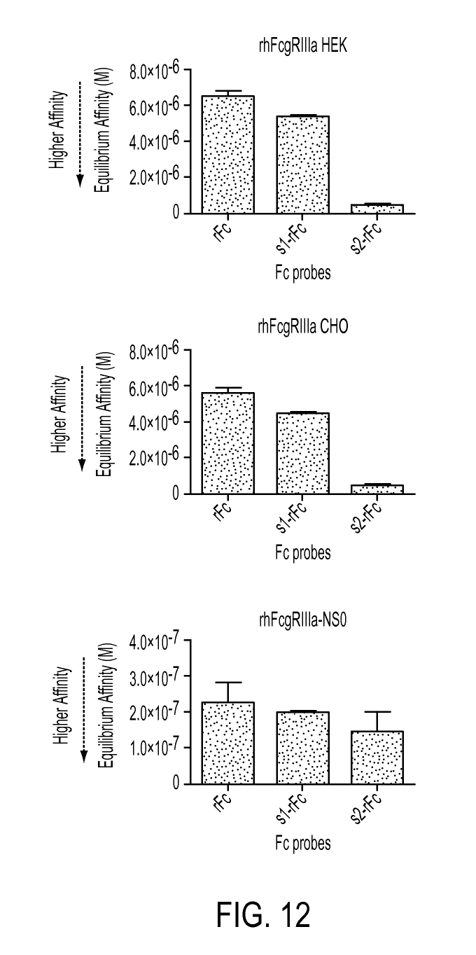

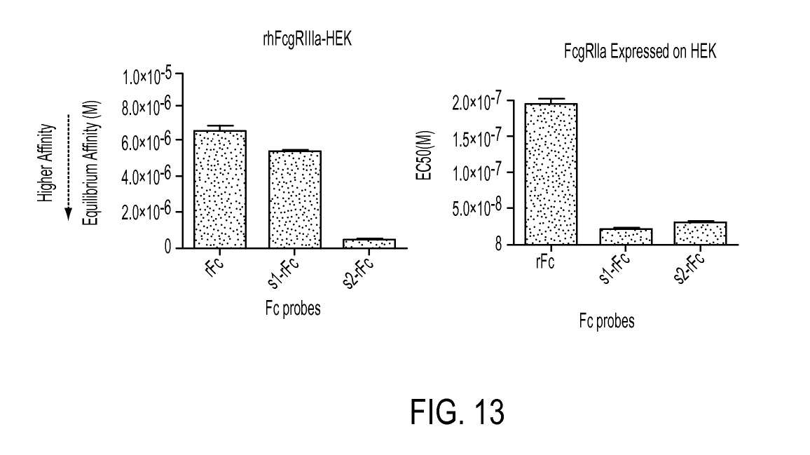

To evaluate the effects of IgG-Fc sialylation on interactions with Fc-receptors and specifically, to determine how the glycoforms of Fc receptors influence the interactions with different Fc-glycoforms, the interactions of sialylated Fc glycoforms with FcγRIIIa were analyzed using SPR binding assays. As shown in FIG. 12, the affinity of the recombinant Fc containing di-sialylated glycoforms (s2-rFc) for FcγRIIIa was higher (by an order of magnitude) regardless of the Fc-receptor glycoform (FIG. 12). In addition, the affinity of Fc sialylated glycoforms for soluble rhFcγRIIIa expressed in HEK cells (measured by SPR direct binding assay) was compared to the relative affinity (EC50) of the Fc glycoforms for FcγRIIIa expressed on the surface of HEK cells (measured by CTRF cellular competition assay). Similar increases in binding of sialylated Fc with FcγRIIIa were observed (FIG. 13). In the cellular competition assay, a more pronounced increase in binding was observed with s1-rFc than was seen in the SPR studies. However, a general trend of sialylated Fc glycoforms having increased affinity for human FcgRIIIa was observed in both assays.

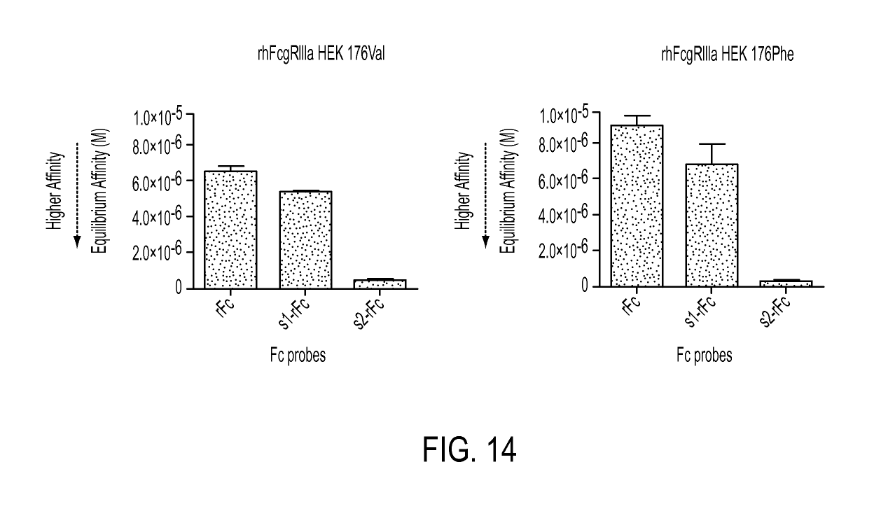

The binding of rFc with FcγRIIIa has been reported to be decreased by approximately 40% when the FcγRIIIa includes a Phe176 polymorphic variation. To determine whether the rFc glycoforms exhibited this reduced affinity to the Phe176 polymorph, SPR binding studies were performed for the Fc glycoforms and either Val176 or Phe176 FcγRIIIa polymorphs. As shown in FIG. 14, rFc demonstrated a reduced affinity for the Phe176 polymorph relative to the Val176 polymorph. Interestingly, neither s1-rFc nor s2-rFc glycoforms exhibited a decreased affinity for the Phe176 glycoform relative to the Val176 glycoform (FIG. 14).

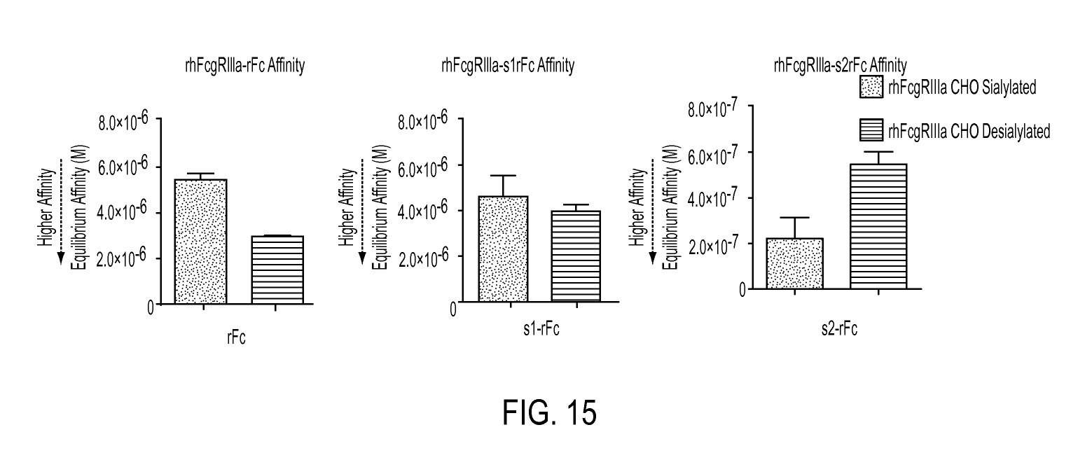

Finally, to compare directly the effect of sialylation of the FcγRIIIa on the binding of different Fc glycoforms, rhFcγRIIIa was expressed in CHO cells, and the binding of rFc, s1-rFc and s2-rFc to expressed FcγRIIIa before and after desialylation of the receptor were compared. Interestingly, rFc bound the desialylated FcγRIIIa with about 80% greater affinity than the sialylated FcγRIIIa form (FIG. 15). However, the binding of recombinant s1-rFc was not affected by the lower sialylation state of the FcγRIIIa receptor, while the affinity for s2-rFc was decreased by approximately 50%.

These data demonstrate that particular Fc glycoforms have specific binding affinities for particular glycoforms of FcγRs (e.g., FcγRIIIa). Further, based on these data, the interactions of Fc-containing glycoproteins and FcγRs can be modified by rationally glycoengineering one or both of Fc-containing glycoproteins and FcγRs.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A glycoprotein composition comprising a glycoform comprising an Fc region comprising a target level of glycans selected from the group consisting of a high mannose glycan, a sialylated glycan, a galactosylated glycan, a glycan comprising a terminal N-acetylglucosamine, a fucosylated glycan, a sulfated glycan, and combinations thereof,
which composition is characterized in that, when it is contacted with an FcγIIIa receptor having a terminal glycan selected from the group consisting of mannose, N-acetylglucosamine, and beta-1,4 galactose, the composition shows an altered affinity for the FcγIIIa receptor as compared with a reference glycoprotein composition lacking the glycoform.

2. The glycoprotein composition of claim 1, wherein the target level of glycans is selected from one or more of:
(a) about 10% to 100% high mannose glycan,
(b) about 10% to 100% sialylated glycan,
(c) about 10% to 100% galactosylated glycan,
(d) about 10% to 100% terminal N-acetylglucosamine,
(e) about 10% to 100% fucosylated glycan, and
(f) about 10% to 100% sulfated glycan.

3. A therapeutic preparation comprising the glycoprotein composition of claim 1 or claim 2, wherein the therapeutic preparation comprises about 10% to about 100% of said glycoform.

4. The composition of claim 1 or 2, comprising a second glycoform comprising a second target level of (a)-(f).

5. The preparation of claim 4, wherein the composition is characterized in that, when it is contacted with an FcγIIIa receptor having a terminal glycan selected from the group consisting of mannose, N-acetylglucosamine, and beta-1,4 galactose, the composition shows an altered affinity for the FcγIIIa receptor as compared with a reference glycoprotein composition lacking one or both of the first and second glycoforms.

6. The glycoprotein composition of claim 1, wherein the composition shows a higher affinity for the FcγIIIa receptor as compared with the reference glycoprotein composition.

7. The glycoprotein composition of claim 1, wherein the composition shows a lower affinity for the FcγIIIa receptor as compared with the reference glycoprotein composition.

8. The glycoprotein composition of claim 1, wherein the reference glycoprotein composition has a level of glycans that is higher than the target level.

9. The glycoprotein composition of claim 1, wherein the reference glycoprotein composition has a level of glycans that is lower than the target level.

10. A glycoprotein composition comprising a glycoform comprising an immunoglobulin Fc region comprising a target level of fucosylated glycan,
which composition is characterized in that, when it is contacted with an FcγIIIa receptor, the composition shows a higher affinity for the FcγIIIa receptor as compared with a reference glycoprotein composition having (a) a lower level of the glycoform or (b) a lower level of fucosylated glycan on the glycoform.

11. The therapeutic preparation of claim 10, wherein the FcγIIIa receptor comprises a terminal glycan selected from the group consisting of mannose, N-acetylglucosamine, and beta-1,4 galactose.

12. A method of producing a therapeutic preparation, comprising:
providing or obtaining an analysis of one or more glycans of an FcγIIIa receptor; and
if the one or more glycans comprise a terminal mannose, a terminal N-acetylglucosamine, or a terminal beta-1,4 galactose, producing a therapeutic preparation comprising a glycoform comprising an immunoglobulin Fc region comprising a target level of glycans selected from the group consisting of a high mannose glycan, a sialylated glycan, a galactosylated glycan, a glycan comprising a terminal N-acetylglucosamine, a sulfated glycan, a fucosylated glycan, and combinations thereof, thereby producing a therapeutic preparation.

13. A method of producing a therapeutic preparation, comprising:
analyzing one or more glycans of an FcγIIIa receptor;
providing a composition of glycoproteins comprising an immunoglobulin Fc region comprising glycans; and
if the one or more glycans of the receptor comprise a terminal mannose, a terminal N-acetylglucosamine, or a terminal beta-1,4 galactose, enriching the composition for a glycoform comprising a target level of glycans selected from the group consisting of a high mannose glycan, a sialylated glycan, a galactosylated glycan, a glycan comprising a terminal N-acetylglucosamine, a sulfated glycan, a fucosylated glycan, and combinations thereof, thereby producing a therapeutic preparation.

14. A method of selecting a subject for treatment with a therapeutic preparation, comprising:
isolating a cell comprising an FcγIIIa receptor from a biological sample of the subject;
analyzing one or more glycans of the FcγIIIa receptor; and
selecting the subject for treatment with a therapeutic preparation if the one or more glycans comprise a terminal mannose, a terminal N-acetylglucosamine, or a terminal beta-1,4 galactose,
wherein the therapeutic preparation comprises a glycoform comprising an immunoglobulin Fc region comprising a target level of glycans selected from the group consisting of a high mannose glycan, a sialylated glycan, a galactosylated glycan, a glycan comprising a terminal N-acetylglucosamine, a sulfated glycan, a fucosylated glycan, and combinations thereof.

15. A method of treating a subject, comprising:
isolating a cell comprising an FcγIIIa receptor from a biological sample of the subject;
analyzing one or more glycans of the FcγIIIa receptor; and
treating the subject with a therapeutic preparation if the one or more glycans comprise a terminal mannose, a terminal N-acetylglucosamine, or a terminal beta-1,4 galactose,
wherein the therapeutic preparation comprises a glycoform comprising an immunoglobulin Fc region comprising a target level of glycans selected from the group consisting of a high mannose glycan, a sialylated glycan, a galactosylated glycan, a glycan comprising a terminal N-acetylglucosamine, a sulfated glycan, a fucosylated glycan, and combinations thereof.

16. A glycoprotein composition comprising a glycoform comprising an Fc region comprising a target level of sialylated glycan,
which composition is characterized in that, when it is contacted with an FcγIIIa receptor having a terminal glycan selected from the group consisting of mannose, N-acetylglucosamine, or beta-1,4 galactose, the composition shows a higher affinity for the FcγIIIa receptor as compared with a reference glycoprotein composition having (a) a lower level of the glycoform or (b) a lower level of sialylated glycan on the glycoform.

17. A glycoprotein composition comprising a glycoform comprising an Fc region comprising a target level of sialylated glycan,
which composition is characterized in that, when it is contacted with a population of FcγIIIa receptors comprising glycans, the composition shows a higher affinity for an FcγIIIa receptor comprising a terminal N-acetylglucosamine as compared with an Fc receptor comprising a terminal mannose or a terminal beta-1,4 galactose.

18. The glycoprotein composition of claim 17, wherein the target level of sialylated glycan is about 30% to about 100%.

19. A glycoprotein composition comprising a glycoform comprising an Fc region comprising a target level of sialylated glycan,
which composition is characterized in that, when it is contacted with a population of FcγIIIa receptors comprising glycans, the composition shows a lower affinity for an FcγIIIa receptor comprising a terminal mannose as compared with an Fc receptor comprising a terminal beta-1,4 galactose or a terminal N-acetylglucosamine.

20. A glycoprotein composition comprising a glycoform comprising an Fc region comprising a target level of sialylated glycan,
which composition is characterized in that, when it is contacted with a population of FcγIIIa receptors comprising glycans, the composition shows a lower affinity for an FcγIIIa receptor comprising a terminal beta-1,4 galactose as compared with an Fc receptor comprising a terminal N-acetylglucosamine.

21. The glycoprotein composition of claim 19 or claim 20, wherein the target level of sialylated glycan is about 0% to about 30%.

22. A therapeutic preparation comprising the glycoprotein composition of any one of claims 16-21, wherein the therapeutic preparation comprises about 10% to about 100% of said glycoform.

23. A method of producing a preparation of glycoproteins, comprising:
providing a plurality of Fcγ receptors;
determining binding of a reference glycoprotein preparation to the plurality of receptors to obtain a reference binding profile;
producing a glycoprotein preparation comprising a plurality of glycoproteins;
determining binding of the glycoprotein preparation to the plurality of Fcγ receptors to obtain a preparation binding profile; and
formulating the preparation into a drug product if the preparation binding profile is at least about 80% identical to the reference binding profile.

24. The method of claim 23, wherein the plurality of Fcγ receptors are provided on an array.

25. The method of claim 24, wherein the array comprises one or more of FcγRI, FcγRIIA, FcγRIIB, FcγRIIIA, FcγRIIIB, FcγRIV, and FcRn receptors.

26. The method of claim 25, wherein the array comprises FcγRIIIA receptors comprising terminal glycans selected from the group consisting of a mannose, a N-acetylglucosamine, or a beta-1,4 galactose.

27. The method of claim 26, wherein the reference binding profile comprises binding affinities of the reference glycoprotein preparation to one or more FcγRIIIA receptors comprising terminal glycans selected from the group consisting of a mannose, a N-acetylglucosamine, or a beta-1,4 galactose, and the preparation binding profile comprises binding affinities of the glycoprotein preparation to the one or more FcγRIIIA receptors.

28. A method of manufacturing a glycoprotein drug product, comprising:
providing or obtaining a test glycoprotein preparation;
acquiring a binding profile of the test glycoprotein preparation for a plurality of FcγRIII glycoforms; and
processing at least a portion of the test glycoprotein preparation into a drug product if the binding profile of the test glycoprotein preparation meets a binding profile of a reference glycoprotein preparation,
thereby manufacturing a drug product.

29. The method of claim 28, wherein the reference binding profile is a binding profile of an FDA approved Fc-containing therapeutic glycoprotein preparation.

30. The method of claim 29, wherein the FDA approved Fc-containing therapeutic glycoprotein preparation is abciximab, adalimumab, alemtuzumab, basiliximab, bevacizumab, cetuximab, certolizumab, daclizumab, eculizumab, efalizumab, gemtuzumab, ibritumomab, infliximab, muromonab, natalizumab, omalizumab, palivizumab, panitumumab, ranibizumab, rituximab, tositumomab, or trastuzumab.

31. A method of manufacturing a glycoprotein drug product, comprising:
providing a host cell that is genetically engineered to express a recombinant Fc region-containing glycoprotein;
culturing the host cell under conditions whereby the cell expresses the recombinant Fc region-containing glycoprotein;
harvesting the recombinant Fc region-containing glycoprotein from the host cell culture to produce a test glycoprotein preparation;
acquiring a binding profile of the test glycoprotein preparation for a plurality of FcγRIII glycoforms; and
processing or directing the processing of at least a portion of the test glycoprotein preparation as a drug product if the preparation meets a binding profile of a reference glycoprotein preparation,
thereby manufacturing a drug product.

32. A method of identifying a patient who has been diagnosed with a disease, for treatment with a therapeutic, Fc region-containing glycoprotein preparation that is approved for treatment of the disease, wherein the improvement comprises: analyzing one or more FcγR glycans from a biological sample of the patient, and identifying the patient for treatment with the preparation if the FcγR glycans match a reference glycan profile.
